SEQUENCE LISTING (4 sequences)

SEQ ID NO: 1 - protein, 9 aa, Artificial Sequence
  To quantify glycopeptides for IgG1
EEQYNSTYR

SEQ ID NO: 2 - protein, 9 aa, Artificial Sequence
  To quantify glycopeptides for IgG2/3
EEQFNSTFR

SEQ ID NO: 3 - protein, 9 aa, Artificial Sequence
  To quantify glycopeptides for IgG3/4
EEQYNSTFR

SEQ ID NO: 4 - protein, 9 aa, Artificial Sequence
  To quantify glycopeptides for IgG3/4
EEQFNSTYR

(19) United States
(12) Patent Application Publication
(10) Pub. No.: US 2023/0365713 A1
(43) Pub. Date: Nov. 16, 2023

(54) SIALYLATED GLYCOPROTEINS

(71) Applicant: Momenta Pharmaceuticals, Inc., Titusville, NJ (US)

(72) Inventor: Siddhesh PATIL, Boston, MA (US)

(21) Appl. No.: 18/022,069

(22) PCT Filed: Aug. 20, 2021
(86) PCT No.: PCT/IB2021/057658

§ 371 Date: Feb. 17, 2023

Related U.S. Application Data
(60) Provisional application No. 63/068,098, filed on Aug. 20, 2020.

(51) Int. Cl. C07K 16/46; A61K 47/26; A61K 47/18; A61K 47/12

(52) U.S. Cl. CPC C07K 16/46; A61K 47/12; A61K 47/183; A61K 47/26; C07K 2317/41; C07K 2317/52

(57) ABSTRACT

Described herein are liquid pharmaceutical compositions comprising immunoglobulins.

### CLAIM OF PRIORITY

This application claims the benefit of U.S. Provisional Application Serial No. 63/068,098, filed on Aug. 20, 2020. The entire contents of the foregoing are incorporated herein by reference.

### TECHNICAL FIELD

Described herein are liquid pharmaceutical compositions comprising immunoglobulins.

### BACKGROUND

The identification of the important role of Fc domain sialylation has presented an opportunity to develop potent immunoglobulin therapies. One commercially available source of immunoglobulins is intravenous immunoglobuin (IVIg), which is prepared from the pooled plasma of human donors (e.g., pooled plasma from at least 1,000 donors) and used to treat a variety of inflammatory disorders. Commercially available IVIg preparations generally exhibit low levels of sialylation on the Fc domain of the antibodies present. Specifically, they exhibit low levels of di-sialylation of the branched glycans on the Fc region. Further, IVIg preparations have distinct limitations, such as variable efficacy, high costs, and limited supply.

### SUMMARY

Described herein are pharmaceutical compositions comprising hypersialylated immunoglobulins (hsIgG). HsIgG has a very high level of sialic acid on the branched glycans on the Fc region of the immunoglobulins, for example, at least 50% (60%, 70%, 80%, 90% or more) of the branched glycans on the Fc region of the immunoglobulins are sialylated via NeuAc-α 2,6-Gal terminal linkages on both the α1,3 arm and the α1,6 arm of the branched glycan. HsIgG contains a diverse mixture of IgG antibodies, primarily IgG1 antibodies. The diversity of the antibodies is high. The immunoglobulins used to prepare hsIgG can be obtained, for example from pooled human plasma (e.g., pooled plasma from at least 1,000 - 30,000 donors).

The pharmaceutical compositions described herein provide pharmaceutically acceptable hsIgG compositions that are stable against a number of stressors associated with shipping (e.g., temperature, agitation, freeze-thaw cycles, and/or photosensitivity). The pharmaceutical compositions described herein provide pharmaceutically acceptable hsIgG compositions that can be shipped and handled in liquid form. The formulations are stable upon dilution, e.g., dilution in 5% dextrose for intravenous administration. The formulations are stable, for example, at 5° C. for at least 7 months and at 25° C. at least one month, for two years at 2-8° C. and/or two weeks at 15-30° C.

Described herein are liquid pharmaceutical compositions comprising immunoglobulins in about 10 mM sodium acetate, about 0.02% (w/v) polysorbate 20, and at least one of about 250 mM glycine or about 5% (w/v) sorbitol, wherein at least 50% of branched glycans on the Fc region of the immunoglobulins are disialylated by way of NeuAc-α 2,6-Gal terminal linkages, wherein the pH of the composition is 4 -7.

In some embodiments, the liquid pharmaceutical composition comprises 250 mM glycine. In some embodiments, the liquid pharmaceutical composition comprises 5% (w/v) sorbitol.

In some embodiments, the concentration of immunoglobulins is 50-275 mg/mL. In some embodiments, the concentration of immunoglobulins is 50-250 mg/mL.

In some embodiments, the liquid pharmaceutical composition comprises 5% (w/v) sorbitol and the concentration of immunoglobulins is 100-275 mg/mL. In some embodiments, the liquid pharmaceutical composition comprises 5% (w/v) sorbitol and the concentration of immunoglobulins is 70-130 mg/mL, 90-110 mg/mL, or 80-120 mg/mL.

In some embodiments, at least 60%, 70%, 80%, 90% or 95% of branched glycans on the Fc domain of the immunoglobulins are disialylated by way of NeuAc-α 2,6-Gal terminal linkages. In some embodiments, at least 60%, 70%, 80%, 90% or 95% of branched glycans on the immunoglobulins are disialylated by way of NeuAc-α 2,6-Gal terminal linkages. In some embodiments, at least 60%, 70%, 80%, 90% or 95% of branched glycans on the Fab domain of the immunoglobulins are disialylated by way of NeuAc-α 2,6-Gal terminal linkages.

In some embodiments, at least 90% of the immunoglobulins are IgG immunoglobulins. In some embodiments, at least 95% of the immunoglobulins are IgG immunoglobulins.

In some embodiments, 5-20% of the immunoglobulins are dimers. In some embodiments, 5-10% of the immunoglobulins are dimers. In some embodiments, at least 80% of the immunoglobulins are monomers or dimers. In some embodiments, at least 85% of the immunoglobulins are monomers or dimers. In some embodiments, at least 90% of the immunoglobulins are monomers or dimers. In some embodiments, 5-20% of the IgG immunoglobulins are dimers. In some embodiments, 5-10% of the IgG immunoglobulins are dimers. In some embodiments, at least 80% of the IgG immunoglobulins are monomers or dimers. In some embodiments, at least 85% of the IgG immunoglobulins are monomers or dimers. In some embodiments, at least 90% of the IgG immunoglobulins are monomers or dimers.

In some embodiments, at least 60%, 70%, 80%, 90% or 95% of branched glycans on the Fc domain of the IgG immunoglobulins are disialylated by way of NeuAc-α 2,6-Gal terminal linkages. In some embodiments, at least 60%, 70%, 80%, 90% or 95% of branched glycans on the IgG immunoglobulins are disialylated by way of NeuAc-α 2,6-Gal terminal linkages. In some embodiments, at least 60%, 70%, 80%, 90% or 95% of branched glycans on the Fab domain of the IgG immunoglobulins are disialylated by way of NeuAc-α 2,6-Gal terminal linkages.

In some embodiments, the pH is 4.0 - 5.5, 4.0 - 4.5, 4.5 - 5.0, 5.0 - 5.5, 4.2 -4.7, 4.7 - 5.3, or 5.1 - 5.3. In some embodiments, the pH is 4.0 - 5.5, 4.0 - 4.5, 4.5 -5.0, 5.0 - 5.5, 4.2 - 4.7, 4.7 - 5.3, or 5.1 - 5.3. In some embodiments, the liquid pharmaceutical composition comprises 5% (w/v) sorbitol and the pH is 5.2 - 5.5 or 5.3 - 5.4. In some embodiments, the pH is about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0.

In some embodiments, the composition has fewer than 1000 particles with a diameter between 10 and 100 micrometers after agitation at 1000 RPM for 8 hours at 2-8° C. In some embodiments, the composition has fewer than 500 particles with a diameter between 10 and 100 micrometers after agitation at 1000 RPM for 8 hours at 2-8° C. In some embodiments, the composition has fewer than 200 particles with a diameter between 10 and 100 micrometers after agitation at 1000 RPM for 8 hours at 2-8° C.

In some embodiments, at least 60%, 70%, 80%, 90% or 95% of branched glycans on the Fc domain of the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22, or 24 months at -70° C., 4° C., 5° C., 25° C., or 40° C. In some embodiments, at least 60%, 70%, 80%, 90% or 95% of branched glycans on the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C. In some embodiments, at least 60%, 70%, 80%, 90% or 95% of branched glycans on the Fab domain of the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C. In some embodiments, 5-10% of the immunoglobulins are dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C. In some embodiments, at least 85% of the immunoglobulins are monomers or dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C. In some embodiments, at least 90% of the immunoglobulins are monomers or dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C.

In some embodiments, the formulation is stable at 5° C. for at least 7 months, at 25° C. for at least one month, 2-8° C. for two years, and/or two weeks at 15-30° C.

In some embodiments, the storage is in a sealed US Pharmacopeia Type 1 glass vial. In some embodiments, the storage is in a sealed 2R Type 1 glass injection vial.

Also described herein is a pre-filled syringe comprising the liquid pharmaceutical composition.

In some embodiments, the liquid pharmaceutical composition is frozen.

Also described herein is a liquid pharmaceutical composition comprising immunoglobulins in about 10 mM sodium acetate, about 0.02% (w/v) polysorbate 20, and at least one of about 250 mM glycine or about 5% (w/v) sorbitol, wherein at least 50% of branched glycans on the immunoglobulins are disialylated by way of NeuAc-α 2,6-Gal terminal linkages, wherein the pH of the composition is 4 - 7.

In some embodiments, the liquid pharmaceutical composition comprises 250 mM glycine. In some embodiments, the liquid pharmaceutical composition comprises 5% (w/v) sorbitol. In some embodiments, the concentration of immunoglobulins is 50-275 mg/mL. In some embodiments, the concentration of immunoglobulins is 50-250 mg/mL. In some embodiments, the liquid pharmaceutical composition comprises 5% (w/v) sorbitol and the concentration of immunoglobulins is 100-275 mg/mL. In some embodiments, the liquid pharmaceutical composition comprises 5% (w/v) sorbitol and the concentration of immunoglobulins is 70-130 mg/mL, 90-110 mg/mL, or 80-120 mg/mL.

In some embodiments, at least 60%, 70%, 80%, 90% or 95% of branched glycans on the immunoglobulins are disialylated by way of NeuAc-α 2,6-Gal terminal linkages. In some embodiments, at least 60%, 70%, 80%, 90% or 95% of branched glycans on the Fc domain of the immunoglobulins are disialylated by way of NeuAc-α 2,6-Gal terminal linkages. In some embodiments, at least 60%, 70%, 80%, 90% or 95% of branched glycans on the Fab domain of the immunoglobulins are disialylated by way of NeuAc-α 2,6-Gal terminal linkages.

In some embodiments, at least 90% of the immunoglobulins are IgG immunoglobulins. In some embodiments, at least 95% of the immunoglobulins are IgG immunoglobulins.

In some embodiments, 5-20% of the immunoglobulins are dimers. In some embodiments, 5-10% of the immunoglobulins are dimers. In some embodiments, at least 80% of the immunoglobulins are monomers or dimers. In some embodiments, at least 85% of the immunoglobulins are monomers or dimers. In some embodiments, at least 90% of the immunoglobulins are monomers or dimers. In some embodiments, 5-20% of the IgG immunoglobulins are dimers. In some embodiments, 5-10% of the IgG immunoglobulins are dimers. In some embodiments, at least 80% of the IgG immunoglobulins are monomers or dimers. In some embodiments, at least 85% of the IgG immunoglobulins are monomers or dimers. In some embodiments, at least 90% of the IgG immunoglobulins are monomers or dimers.

In some embodiments, at least 60%, 70%, 80%, 90% or 95% of branched glycans on the the IgG immunoglobulins are disialylated by way of NeuAc-α 2,6-Gal terminal linkages. In some embodiments, at least 60%, 70%, 80%, 90% or 95% of branched glycans on the Fc domain of the IgG immunoglobulins are disialylated by way of NeuAc-α 2,6-Gal terminal linkages. In some embodiments, at least 60%, 70%, 80%, 90% or 95% of branched glycans on the Fab domain of the IgG immunoglobulins are disialylated by way of NeuAc-α 2,6-Gal terminal linkages.

In some embodiments, the pH is 4.0 - 5.5, 4.0 - 4.5, 4.5 - 5.0, 5.0 - 5.5, 4.2 -4.7, 4.7 - 5.3, or 5.1 - 5.3. In some embodiments, the pH is 4.0 - 5.5, 4.0 - 4.5, 4.5 -5.0, 5.0 - 5.5, 4.2 - 4.7, 4.7 - 5.3, or 5.1 - 5.3. In some embodiments, the liquid pharmaceutical composition comprises 5% (w/v) sorbitol and the pH is 5.2 - 5.5 or 5.3 - 5.4. In some embodiments, the pH is about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0.

In some embodiments, the composition has fewer than 1000 particles with a diameter between 10 and 100 micrometers after agitation at 1000 RPM for 8 hours at 2-8° C. In some embodiments, the composition has fewer than 500 particles with a diameter between 10 and 100 micrometers after agitation at 1000 RPM for 8 hours at 2-8° C. In some embodiments, the composition has fewer than 200 particles with a diameter between 10 and 100 micrometers after agitation at 1000 RPM for 8 hours at 2-8° C.

In some embodiments, at least 60%, 70%, 80%, 90% or 95% of branched glycans on the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22, or 24 months at -70° C., 4° C., 5° C., 25° C., or 40° C. In some embodiments, at least 60%, 70%, 80%, 90% or 95% of branched glycans on the Fc region of the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C. In some embodiments, at least 60%, 70%, 80%, 90% or 95% of branched glycans on the Fab domain of the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C.

In some embodiments, 5-10% of the immunoglobulins are dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C.

In some embodiments, at least 85% of the immunoglobulins are monomers or dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C. In some embodiments, at least 90% of the immunoglobulins are monomers or dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C. In some embodiments, the formulation is stable at 5° C. for at least 7 months, at 25° C. for at least one month, 2-8° C. for two years, and/or two weeks at 15-30° C.

In some embodiments, the storage is in a sealed US Pharmacopeia Type 1 glass vial. In some embodiments, the storage is in a sealed 2R Type 1 glass injection vial.

Also described herein is a pre-filled syringe comprising the liquid pharmaceutical composition.

In some embodiments, the liquid pharmaceutical composition is frozen.

Also described herein are methods for treating a disorder, the method comprising administering the liquid pharmaceutical composition of any of the forgoing claims at a dose that is 1%-10% of the effective dose of IVIG for treating the disorder.

In some embodiments, the hsIgG preparation is administered at a dose of 5 mg/kg to 100 mg/kg.

In some embodiments, the disorder is an inflammatory disorder. In some embodiments, the subject is suffering from antibody deficiency. In some embodiments, the subject is suffering from primary antibody deficiency. In some embodiments, the disorder is associated with the presence of autoantibodies.

In some embodiments, the dose of hsIVIG is as effective as the effective dose of IVIG. In some embodiments, the hsIgG preparation is administered at the same frequency as the effective dose of IVIG.

In some embodiments, the disorder is a neurological disorder. In some embodiments, the neurological disorder is selected from the group consisting of: dermatomyositis, Guillain-Barre syndrome, chronic inflammatory demyelinating polyneuropathy (CIDP), multifocal motor neuropathy (MMN), myasthenia gravis and stiff person syndrome.

In some embodiments, the disorder is selected from the group consisting of: immune cytopenias, parvovirus B19 associated red cell aplasia, hypogammaglobulinaemia secondary to myeloma and chronic lymphatic leukaemia and post-bone marrow transplantation.

In some embodiments, the disorder is selected from the group consisting of: sculitis, systemic lupus erythematosis (SLE), mucous membrane pemphigoid and uveitis and in dermatology it is used most commonly to treat Kawasaki syndrome, dermatomyositis, toxic epidermal necrolysis and the blistering diseases.

In some embodiments, the disorder is FDA-approved for treatment with IVIG or IVIG is indicated for treatment of the disorder.

In some embodiments, the hsIgG preparation preparation is 1% - 10% of the FDA approved IVIG dose for the disorder.

In some embodiments, the disorder is selected from the group consisting of: Myocarditis, Acute motor axonal neuropathy, Adiposis dolorosa, Anti-Glomerular Basement Membrane nephritis; Goodpasture syndrome, Antiphospholipid syndrome (APS, APLS), Antisynthetase syndrome; Myositis, ILD, ataxic neuropathy (acute & chronic), Autoimmune enteropathy (AIE), Autoimmune neutropenia, Autoimmune retinopathy, Autoimmune thyroiditis, Autoimmune urticaria, Dermatitis herpetiformis, Epidermolysis bullosa acquisita, Essential mixed cryoglobulinemia, Granulomatosis with polyangiitis(GPA), Mixed connective tissue disease(MCTD), Neuromyotonia, Optic neuritis, Paraneoplastic cerebellar degeneration, Anti-N-Methyl-D-Aspartate (Anti-NMDA) Receptor Encephalitis, Autoimmune hemolytic anemia, Autoimmune thrombocytopenic purpura, Chronic inflammatory demyelinating polyneuropathy, Dermatomyositis, Gestational pemphigoid, Graves’ disease, Guillain-Barré syndrome, IgG4-related disease, Lambert-Eaton myasthenic syndrome, Lupus nephritis, Myositis, Multifocal motor neuropathy, Myasthenia gravis, Neuromyelitis optica, Pemphigus vulgaris, Polymyositis, Systemic Lupus Erythematosus (SLE), and combinations thereof.

In some embodiments, the disorder is selected from the group consisting of: Acute disseminated encephalomyelitis (ADEM), Autoimmune Angioedema (Acquired angioedema type II), Autoimmune hepatitis (Type I & Type II), Autoimmune hypophysitis; Lymphocytic hypophysitis, Autoimmune inner ear disease (AIED), Evans syndrome, Graves ophthalmopathy, Hashimoto’s encephalopathy, IgA vasculitis (IgAV), Latent autoimmune hepatitis, Linear IgA disease (LAD), Lupus vasculitis, Membranous glomerulonephritis, Microscopic polyangiitis (MPA), Mooren’s ulcer, Morphea, Opsoclonus myoclonus syndrome, Ord’s thyroiditis, Palindromic rheumatism, Paraneoplastic opsoclonus - myoclonus-ataxia with neuroblastoma, Pediatric Autoimmune Neuropsychiatric Disorder Associated with Streptococcus (PANDAS), Postpericardiotomy syndrome, Primary biliary cirrhosis (PBC), Rasmussen Encephalitis, Rheumatoid vasculitis, Schnitzler syndrome, Sydenham chorea, Undifferentiated connective tissue disease (UCTD), Miller Fisher Syndrome, and combinations thereof.

Also described herein are methods of treating CIDP in a subject having CIDP comprising administering a hsIgG preparation at an effective dose than is 10% of or less than 10% of the effective dose for IVIG. In some embodiments, the effective dose for treating CIDP with IVIG is 200-2000 mg/kg. In some embodiments, the hsIgG preparation is administered at an effective dose that is 10% of or less than the effective dose for treating CIDP IVIG. In some embodiments, the hsIgG preparation is administered at a dose of 1% of the effective dose for treating CIDP IVIG. In some embodiments, the hsIgG preparation is administered at a dose of about 2, 3, 4, 5, 6, 7, 8, 9, 10, 15, 20, 25, 30, 35, 40, 45, 50, 55, 60, 65, 70, 75, 80, 90, 100, 110, 120, 130, 140, 150, 160, 170, 180, 190, or 200 mg/kg.

Also described herein are methods of treating ITP in a subject having ITP comprising administering a hsIgG preparation at an effective dose that is 10% of or less than 10% of the effective dose for IVIG. In some embodiments, the effective dose for treating ITP with IVIG is 1000-2000 mg/kg mg/kg. In some embodiments, the hsIgG preparation is administered at an effective dose that is 10% of or less than the effective dose for treating ITP with IVIG. In some embodiments, the hsIgG preparation is administered at a dose of 1%-5% of the effective dose for treating ITP with IVIG. In some embodiments, the hsIgG preparation is administered at a dose of about 10, 20, 30, 40, 50, 60, 70, 80, 90, 100, 110, 120, 130, 140, 150, 160, 170, 180, 190, or 200 mg/kg.

Also described herein are methods of treating wAIHA in a subject having wAIHA comprising administering a hsIgG preparation at an effective dose that is 10% of or less than 10% of the of the effective dose for IVIG.

In some embodiments, the effective dose for treating wAIHA with IVIG is 1000 mg/kg. In some embodiments, the hsIgG preparation is administered at a dose of less than 10% of the effective dose for treating wAIHA with IVIG. In some embodiments, the hsIgG preparation is administered at a dose of 1%-5% of the effective dose for treating wAIHA with IVIG. In some embodiments, the hsIgG preparation is administered at a dose of about 10, 20, 30, 40, 50, 60, 70, 80, 90, or 100 mg/kg.

Also described herein are methods of treating Guillain-Barre Syndrome in a subject having Guillain-Barre Syndrome comprising administering a hsIgG preparation at an effective dose that is 10% of or less than 10% of the of the effective dose for IVIG. In some embodiments, the effective dose for treating Guillain-Barre Syndrome with IVIG is 1000-2000 mg/kg. In some embodiments, the hsIgG preparation is administered at a dose of less than 10% of the effective dose for treating Guillain-Barre Syndrome with IVIG. In some embodiments, the hsIgG preparation is administered at a dose of 1%-5% of the effective dose for treating Guillain-Barre Syndrome with IVIG. In some embodiments, the hsIgG preparation is administered at a dose of about 10, 20, 30, 40, 50, 60, 70, 80, 90, 100, 110, 120, 130, 140, 150, 160, 170, 180, 190, or 200 mg/kg.

Also described herein are methods of treating PID (primary humoral immunodeficiency disease) in a subject having PID comprising administering the hsIgG preparation at an effective dose that is 10% of or less than 10% of the of the effective dose for IVIG. In some embodiments, the effective dose for treating PID with IVIG is 200-800 mg/kg. In some embodiments, the hsIgG preparation is administered at a dose of less than 10% of the effective dose for treating PID with IVIG. In some embodiments, the hsIgG preparation is administered at a dose of 1%-5% of the effective dose for treating PID with IVIG. In some embodiments, the hsIgG preparation is administered at a dose of about 2, 3, 4, 5, 6, 7, 8, 9, 10, 15, 20, 25, 30, 35, 40, 45, 50, 55, 60, 65, 70, 75, or 80 mg/kg.

Also described herein are methods of treating Kawasaki disease in a subject having Kawasaki disease comprising administering a hsIgG preparation at an effective dose that is 10% of or less than 10% of the of the effective dose for IVIG.

In some embodiments, the effective dose for treating Kawasaki disease with IVIG is 1000-2000 mg/kg. In some embodiments, the hsIgG preparation is administered at a dose of less than 10% of the effective dose for treating Kawasaki disease with IVIG. In some embodiments, the hsIgG preparation is administered at a dose of 1%-5% of the effective dose for treating Kawasaki disease with IVIG. In some embodiments, the hsIgG preparation is administered at a dose of about 10, 20, 30, 40, 50, 60, 70, 80, 90, 100, 110, 120, 130, 140, 150, 160, 170, 180, 190, or 200 mg/kg.

In some embodiments of any of the methods described herein, the dose of the pharmaceutical composition has similar efficacy to the effective dose for IVIG.

In some embodiments of any of the methods described herein, at least one side effect attributed to the effective dose for IVIG is alleviated by administering the pharmaceutical composition.

In some embodiments of any of the methods described herein, the pharmaceutical composition is administered subcutaneously.

Also described herein is a syringe suitable for subcutaneous injection comprising 2 mL or less than the pharmaceutical composition of any one of the preceding claims.

Also described herein is a liquid pharmaceutical composition comprising immunoglobulins in 10 mM sodium acetate and at least one of about 250 mM glycine or about 5% (w/v) sorbitol, wherein at least 50% of branched glycans on the Fc region of the immunoglobulins are disialylated by way of NeuAc-α 2,6-Gal terminal linkages wherein the pH of the composition is 4 - 7. In some embodiments, the liquid pharmaceutical composition comprises immunoglobulins in 10 mM sodium acetate. In some embodiments, the liquid pharmaceutical composition further comprises 0.02% (w/v) polysorbate 20. In some embodiments, the liquid pharmaceutical composition comprises 250 mM glycine. In some embodiments, the liquid pharmaceutical composition further comprises 5% (w/v) sorbitol. In some embodiments, the pH is 4.0 - 5.5, 4.0 - 4.5, 4.5 - 5.0, 5.0 - 5.5, 4.2 - 4.8, 4.7 - 5.3, or 5.1 - 5.3 In some embodiments, the pH is about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0.

In various embodiments: the concentration of immunoglobulins is 50-250 mg/mL; the concentration of immunoglobulins is 70-130 mg/mL; the concentration of immunoglobulins is 80-120 mg/mL; the concentration of immunoglobulins is 90-110 mg/mL; at least 60%, 70%, 80%, 90% or 95% of branched glycans on the Fc region of the immunoglobulins are disialylated by way of NeuAc-α 2,6-Gal terminal linkages; at least 60%, 70%, 80%, 90% or 95% of branched glycans on the immunoglobulins are disialylated by way of NeuAc-α 2,6-Gal terminal linkages; at least 60%, 70%, 80%, 90% or 95% of branched glycans on the Fab region of the immunoglobulins are disialylated by way of NeuAc-α 2,6-Gal terminal linkages; at least 90% of the immunoglobulins are IgG immunoglobulins; at least 95% of the immunoglobulins are IgG immunoglobulins; 5-20% of the immunoglobulins are dimers; 5-10% of the immunoglobulins are dimers; at least 80% of the immunoglobulins are monomers or dimers; at least 85% of the immunoglobulins are monomers or dimers; at least 90% of the immunoglobulins are monomers or dimers; 5-20% of the IgG immunoglobulins are dimers; 5-10% of the IgG immunoglobulins are dimers; at least 80% of the IgG immunoglobulins are monomers or dimers; at least 85% of the IgG immunoglobulins are monomers or dimers; at least 90% of the IgG immunoglobulins are monomers or dimers; at least 60%, 70%, 80%, 90% or 95% of branched glycans on the Fc region of the IgG immunoglobulins are disialylated by way of NeuAc-α 2,6-Gal terminal linkages; at least 60%, 70%, 80%, 90% or 95% of branched glycans on the IgG immunoglobulins are disialylated by way of NeuAc-α 2,6-Gal terminal linkages; at least 60%, 70%, 80%, 90% or 95% of branched glycans on the Fab region of the IgG immunoglobulins are disialylated by way of NeuAc-α 2,6-Gal terminal linkages; the composition has fewer than 1000 particles with a diameter between 10 and 100 micrometers after agitation at 1000 RPM for 8 hours at 2-8° C.; the composition has fewer than 500 particles with a diameter between 10 and 100 micrometers after agitation at 1000 RPM for 8 hours at 2-8° C.; the composition has fewer than 200 particles with a diameter between 10 and 100 micrometers after agitation at 1000 RPM for 8 hours at 2-8° C.; at least 60%, 70%, 80%, 90% or 95% of branched glycans on the Fc region of the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C. - 40° C., -70° C. - 25° C., 0° C. - 5° C., 0° C. - 25° C., or 0° C. - 40° C., or about -70° C., 4° C., 5° C., 25° C., or 40° C.; at least 60%, 70%, 80%, 90% or 95% of branched glycans on the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C. - 40° C., -70° C. - 25° C., 0° C. - 5° C., 0° C. -25° C., or 0° C. - 40° C., or about -70° C., 4° C., 5° C., 25° C., or 40° C.; at least 60%, 70%, 80%, 90% or 95% of branched glycans on the Fab region of the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C. - 40° C., -70° C. - 25° C., 0° C. - 5° C., 0° C. - 25° C., or 0° C. - 40° C., or about -70° C., 4° C., 5° C., 25° C., or 40° C.; 5-10% of the immunoglobulins are dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C. - 40° C., -70° C. - 25° C., 0° C. - 5° C., 0° C. - 25° C., or 0° C. - 40° C., or about -70° C., 4° C., 5° C., 25° C., or 40° C.; at least 85% of the immunoglobulins are monomers or dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C. - 40° C., -70° C. - 25° C., 0° C. - 5° C., 0° C. - 25° C., or 0° C. - 40° C., or about -70° C., 4° C., 5° C., 25° C., or 40° C.; at least 90% of the immunoglobulins are monomers or dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C. - 40° C., -70° C. - 25° C., 0° C. - 5° C., 0° C. - 25° C., or 0° C. - 40° C., or about -70° C., 4° C., 5° C., 25° C., or 40° C.; the storage is in a sealed US Pharmacopeia Type 1 glass vial; the storage is in a sealed 2R Type 1 glass injection vial.

In hsIgG at least 50% (e.g., 60%, 70%, 80%, 82%, 85%, 87%, 90%, 92%, 94%, 95%, 97%, 98% up to and including 100%) of branched glycans on the Fc region of the immunoglobulins have a sialic acid residue on both the α 1,3 arm and the α 1,6 arm (i.e., are disialylated by way of NeuAc-α 2,6-Gal terminal linkages). In some embodiments, in addition to the Fc sialylation, at least 50% (e.g., 60%, 70%, 80%, 82%, 85%, 87%, 90%, 92%, 94%, 95%, 97%, 98% or up to and including 100%) of branched glycans on the Fab region are disialylated by way of NeuAc-α 2,6-Gal terminal linkages. In some cases, at least 85%, (87%, 90%, 92%, 94%, 95%, 97%, 98% or up to and including 100%) of total branched glycans (sum of glycans on the Fc and Fab domains) are disialylated by way of NeuAc-α 2,6-Gal terminal linkages. In some embodiments, less than 50% (e.g., less than 40%, 30%, 20%, 15%, 10%, 5%, 4%, 3%, 2%, 1%) of branched glycans on the Fc region are mono-sialylated (e.g., sialylated only on the α 1,3 arm or the α 1,6 arm) by way of a NeuAc-α 2,6-Gal terminal linkage. HsIgG preparations are primarily IgG antibodies (e.g., at least 80%, 85%, 90%, 95% wt/wt of the immunoglobulins are IgG antibodies of various isotypes).

As used herein, the term “Fc region” refers to a dimer of two “Fc polypeptides,” each “Fc polypeptide” including the constant region of an antibody excluding the CH1 domain. In some embodiments, an “Fc region” includes two Fc polypeptides linked by one or more disulfide bonds, chemical linkers, or peptide linkers. “Fc polypeptide” refers to the last two constant region immunoglobulin domains of IgA, IgD, and IgG, and the last three constant region immunoglobulin domains of IgE and IgM, and may also include part or the entire flexible hinge N-terminal to these domains.

As used herein, “glycan” is a sugar, which can be monomers or polymers of sugar residues, such as at least three sugars, and can be linear or branched. A “glycan” can include natural sugar residues (e.g., glucose, N-acetylglucosamine, N-acetyl neuraminic acid, galactose, mannose, fucose, hexose, arabinose, ribose, xylose, etc.) and/or modified sugars (e.g., 2′-fluororibose, 2′-deoxyribose, phosphomannose, 6′sulfo N-acetylglucosamine, etc.). The term “glycan” includes homo and heteropolymers of sugar residues. The term “glycan” also encompasses a glycan component of a glycoconjugate (e.g., of a polypeptide, glycolipid, proteoglycan, etc.). The term also encompasses free glycans, including glycans that have been cleaved or otherwise released from a glycoconjugate.

As used herein, the term “glycoprotein” refers to a protein that contains a peptide backbone covalently linked to one or more sugar moieties (i.e., glycans). The sugar moiety(ies) may be in the form of monosaccharides, disaccharides, oligosaccharides, and/or polysaccharides. The sugar moiety(ies) may comprise a single unbranched chain of sugar residues or may comprise one or more branched chains. Glycoproteins can contain O-linked sugar moieties and/or N-linked sugar moieties.

IVIg is a preparation of pooled, polyvalent immunoglobulins, including all four IgG isotypes, extracted from plasma of at least 1,000 human donors. Among the forms of IVIg approved for use in the United States are Gammagard (Baxter Healthcare Corporation), Gammaplex (Bio Products Laboratory), Bivigam (Biotest Pharmaceuticals Corporation), Carimmune NF (CSL Behring AG), Gamunes-C (Grifols Therapeutics, Inc.) Glebogamma DID (Instituto Grifols, SA) and Octagam (Octapharma Pharmazeutika Produktionsges Mbh). IVIg is approved as a plasma protein replacement therapy for immune deficient patients and for other uses. The level of IVIg Fc glycan sialylation varies among IVIg preparations, but is generally less than 20%. The level of disialylation is generally far lower.

As used herein, an “N-glycosylation site of an Fc polypeptide” refers to an amino acid residue within an Fc polypeptide to which a glycan is N-linked. In some embodiments, an Fc region contains a dimer of Fc polypeptides, and the Fc region comprises two N-glycosylation sites, one on each Fc polypeptide.

As used herein “percent (%) of branched glycans” refers to the number of moles of glycan X relative to total moles of glycans present, wherein X represents the glycan of interest.

The term “pharmaceutically effective amount” or “therapeutically effective amount” refers to an amount (e.g., dose) effective in treating a patient, having a disorder or condition described herein. It is also to be understood herein that a “pharmaceutically effective amount” may be interpreted as an amount giving a desired therapeutic effect, either taken in one dose or in any dosage or route, taken alone or in combination with other therapeutic agents.

“Pharmaceutical preparations” and “pharmaceutical products” can be included in kits containing the preparation or product and instructions for use.

“Pharmaceutical preparations” and “pharmaceutical products” generally refer to compositions in which the final predetermined level of sialylation has been achieved, and which are free of process impurities. To that end, “pharmaceutical preparations” and “pharmaceutical products” are substantially free of ST6Gal sialyltransferase and/or sialic acid donor (e.g., cytidine 5′-monophospho-N-acetyl neuraminic acid) or the byproducts thereof (e.g., cytidine 5′-monophosphate).

“Pharmaceutical preparations” and “pharmaceutical products” are generally substantially free of other components of a cell in which the glycoproteins were produced (e.g., the endoplasmic reticulum or cytoplasmic proteins and RNA), if recombinant.

By “purified” (or “isolated”) refers to a polynucleotide or a polypeptide that is removed or separated from other components present in its natural environment. For example, an isolated polypeptide is one that is separated from other components of a cell in which it was produced (e.g., the endoplasmic reticulum or cytoplasmic proteins and RNA). An isolated polynucleotide is one that is separated from other nuclear components (e.g., histones) and/or from upstream or downstream nucleic acids. An isolated polynucleotide or polypeptide can be at least 60% free, or at least 75% free, or at least 90% free, or at least 95% free from other components present in natural environment of the indicated polynucleotide or polypeptide.

As used herein, the term “sialylated” refers to a glycan having a terminal sialic acid. The term “mono-sialylated” refers to branched glycans having one terminal sialic acid, e.g., on an α1,3 arm or an α1,6 arm. The term “disialylated” refers to a branched glycan having a terminal sialic acid on two arms, e.g., both an α1,3 arm and an α1,6 arm.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, the term “about” a number refers to that number plus or minus 10% of that number. The term “about” a range refers to that range minus 10% of its lowest value and plus 10% of its greatest value.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 schematically depicts an example of a branched glycan. Light circles are Gal; dark circles are Man; triangles are Fuc, diamonds are NANA; and squares are GlcNAc.

FIG. 2 is a schematic representation of enzymatic sialylation reaction to transform pooled immunoglobulins to hsIgG (left) and IgG Fc glycan profile for the starting IVIg and for hsIgG enzymatically prepared from IVIg (right). Glycan profiles for the different IgG subclasses are derived via glycopeptide mass spectrometry analysis. Peptide sequences used to quantify glycopeptides for different IgG subclasses are: IgG1 = EEQYNSTYR (SEQ ID NO:1), IgG2/3 EEQFNSTFR (SEQ ID NO:2), IgG3/4 EEQYNSTFR (SEQ ID NO:3) and EEQFNSTYR (SEQ ID NO″4).

FIG. 3 shows aggregation curves overlaid for solutions of M254 of varying concentrations from 100 mg/mL to 250 mg/mL as measured using scattering light intensity at 473 nm over a thermal ramp from 20° C. to 90° C.

### DETAILED DESCRIPTION

Immunoglobulins are glycosylated at conserved positions in the constant regions of their heavy chain. For example, human IgG has a single N-linked glycosylation site at Asn297 of the CH2 domain. Each immunoglobulin type has a distinct variety of N-linked carbohydrate structures in the constant regions. For human IgG, the core oligosaccharide normally consists of GlcNAc₂Man₃GlcNAc, with differing numbers of outer residues. Variation among individual IgG’s can occur via attachment of galactose and/or galactose-sialic acid at one or both terminal GlcNAc or via attachment of a third GlcNAc arm (bisecting GlcNAc).

The present disclosure encompasses, in part, pharmaceutical preparations including pooled human immunoglobulins having an Fc region having particular levels of branched glycans that are sialylated on both of the branched glycans in the Fc region (e.g., with a NeuAc-α 2,6-Gal terminal linkage).

### Immunoglobulins

Preparations of pooled, polyvalent human immunoglobulins, including IVIg preparations, are highly complex because they are highly heterogeneous in several regards. They include immunoglobulins pooled from many hundreds or more than 1000 individuals. While at least about 90% or 95% of immunoglobulins are IgG isotype (of all subclasses), other isotypes, including IgA and IgM are present. The immunoglobulins in IVIg and preparations of pooled, polyvalent human immunoglobulins vary in both specificity and glycosylation pattern.

Hypersialylation of pooled, polyvalent immunoglobulins alters the glycans which are present on the immunoglobulins. For some glycans, the alteration entails the addition of one of more galactose molecules and the addition of one or more sialic acid molecules. For other glycans, the alteration entails only the addition of one or more sialic acid molecules. Moreover, while essentially all IgG antibodies, the predominant immunoglobulins in preparations of pooled, polyvalent immunoglobulins, have a glycosylation site on each polypeptide forming Fc region, not all IgG antibodies have a glycosylation site on the Fab domain. Altering the glycosylation of an immunoglobulin preparation alters the structure and activity of the individual immunoglobulins in the preparation and, importantly, alters the interactions between individual immunoglobulins as well as the bulk behavior of preparations of the immunoglobulins.

The widely used formulation used for IVIg preparations is wholly unsuitable for pharmaceutical preparations of hypersialylated immunoglobulins (hsIgG) for at least the reason that the formulations, when used for hsIgG, are not stable to shear stress that occurs in normal shipping of pharmaceutical formulations. When subjected to this type of shear stress, subvisible particles formed in the hsIgG formulations. It is known that such subvisible particles in antibody preparations can cause serious adverse events at the site of injection and off target immune responses. Subvisible particles in antibody preparations can also activate the complement system, cause embolisms, and other negative immunogenic reactions. It was found that the addition of non-ionic surfactants rendered the hsIgG preparations more stable to shear stress and greatly reduced the formation of subvisible particles.

Naturally derived polypeptides that can be used to prepare hsIgG include, for example, immunoglobulins isolated from pooled human serum. HsIgG can also be prepared from IVIg and polypeptides derived from IVIg. HsIgG can be prepared as described in WO2014/179601. Preparation of hsIgG is also described in Washburn et al (Proc Natl Acad Sci U S A. 2015 Mar 17;112(11):E1297-306).The level of sialylation in a hsIgG preparation can be measured on the Fc domain (e.g., the number of branched glycans that are sialylated on an α1,3 arm, an α1,6 arm, or both, of the branched glycans in the Fc domain), or on the overall sialylation (e.g., the number or percentage of branched glycans that are sialylated on an α1,3 arm, an α1,6 arm, or both, of the branched glycans in the preparation of polypeptides whether on the Fc domain or the Fab domain).

Is some cases, the pooled serum used as a source of immunoglobulins for preparing hsIgG is isolated from a specific population of individuals, for example, individuals that produce antibodies against one or more virus, such as COVID-19, SARS, parainfluenza, influenza, but do not have an active infection. In some cases, the immunoglobulins are isolated from a population of individuals in which greater than 50%, 55%, 60%, 75% produce antibodies to a selected virus.

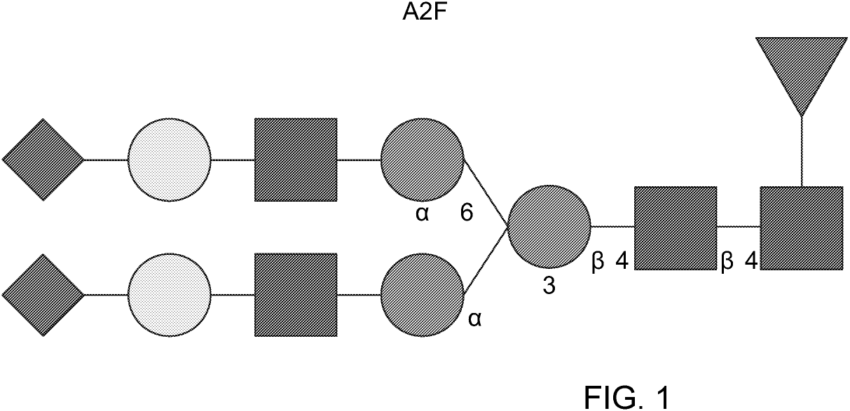

N-linked oligosaccharide chains are added to a protein in the lumen of the endoplasmic reticulum. Specifically, an initial oligosaccharide (typically 14-sugar) is added to the amino group on the side chain of an asparagine residue contained within the target consensus sequence of Asn-X-Ser/Thr, where X may be any amino acid except proline. The structure of this initial oligosaccharide is common to most eukaryotes, and contains three glucose, nine mannose, and two N-acetylglucosamine residues. This initial oligosaccharide chain can be trimmed by specific glycosidase enzymes in the endoplasmic reticulum, resulting in a short, branched core oligosaccharide composed of two N-acetylglucosamine and three mannose residues. One of the branches is referred to in the art as the “α 1,3 arm,” and the second branch is referred to as the “α 1,6 arm,” as shown in FIG. 1.

N-glycans can be subdivided into three distinct groups called “high mannose type,” “hybrid type,” and “complex type,” with a common pentasaccharide core (Man (α 1,6)-(Man(α 1,3))-Man(β 1,4)-GlcpNAc(β 1,4)-GlcpNAc(β 1,N)-Asn) occurring in all three groups.

After initial processing in the endoplasmic reticulum, the polypeptide is transported to the Golgi where further processing may take place. If the glycan is transferred to the Golgi before it is completely trimmed to the core pentasaccharide structure, it results in a “high-mannose glycan.”

Additionally or alternatively, one or more monosaccharies units of N-acetylglucosamine may be added to the core mannose subunits to form a “complex glycan.” Galactose may be added to the N-acetylglucosamine subunits, and sialic acid subunits may be added to the galactose subunits, resulting in chains that terminate with any of a sialic acid, a galactose or an N-acetylglucosamine residue. Additionally, a fucose residue may be added to an N-acetylglucosamine residue of the core oligosaccharide. Each of these additions is catalyzed by specific glycosyl transferases.

“Hybrid glycans” comprise characteristics of both high-mannose and complex glycans. For example, one branch of a hybrid glycan may comprise primarily or exclusively mannose residues, while another branch may comprise N-acetylglucosamine, sialic acid, galactose, and/or fucose sugars.

Sialic acids are a family of 9-carbon monosaccharides with heterocyclic ring structures. They bear a negative charge via a carboxylic acid group attached to the ring as well as other chemical decorations including N-acetyl and N-glycolyl groups. The two main types of sialyl residues found in polypeptides produced in mammalian expression systems are N-acetyl-neuraminic acid (NeuAc) and N-glycolylneuraminic acid (NeuGc). These usually occur as terminal structures attached to galactose (Gal) residues at the non-reducing termini of both N- and O-linked glycans. The glycosidic linkage configurations for these sialyl groups can be either α 2,3 or α 2,6.

Fc regions are glycosylated at conserved, N-linked glycosylation sites. For example, each heavy chain of an IgG antibody has a single N-linked glycosylation site at Asn297 of the C_{H}2 domain. IgA antibodies have N-linked glycosylation sites within the C_{H}2 and C_{H}3 domains, IgE antibodies have N-linked glycosylation sites within the C_{H}3 domain, and IgM antibodies have N-linked glycosylation sites within the C_{H}1, C_{H}2, C_{H}3, and C_{H}4 domains.

Each antibody isotype has a distinct variety of N-linked carbohydrate structures in the constant regions. For example, IgG has a single N-linked biantennary carbohydrate at Asn297 of the C_{H}2 domain in each Fc polypeptide of the Fc region, which also contains the binding sites for C1q and FcγR. For human IgG, the core oligosaccharide normally consists of GlcNAc₂Man₃GlcNAc, with differing numbers of outer residues. Variation among individual IgG can occur via attachment of galactose and/or galactose-sialic acid at one or both terminal GlcNAc or via attachment of a third GlcNAc arm (bisecting GlcNAc).Glycans of polypeptides can be evaluated using any methods known in the art. For example, sialylation of glycan compositions (e.g., level of branched glycans that are sialylated on an α1,3 arm and/or an α1,6 arm) can be characterized using methods described in WO2014/179601.

### Immunoglobulin Compositions

Composition containing hsIgG can include, in addition to antibody monomer, dimers and aggregates of antibodies. In some cases, pH can be used to modulate the percent monomer, dimer, and aggregate in the composition as measured by weight % purity by size exclusion chromatography.

In some cases, lowering the pH increases the weight % of monomer + dimer in the solution. In some cases, lowering the pH increases the weight % monomer in the solution. In some cases, increasing the pH lowers the % monomer in the solution.

In some cases, the weight % aggregate is less than or equal to 3.0% wt/wt (e.g., less than or equal to 2.7, 2.5, 2.3, 2.0, 1.7, 1.5, 1.3, 1.0, 0.9, 0.8, 0.7, 0.6, 0.5, 0.4, 0.3, 0.2, or 0.1 % wt/wt).

In some cases, the weight % Monomer + Dimer is greater than or equal to 97.0% wt/wt (e.g., greater than or equal to 98% wt/wt or 99% wt/wt). In some cases, the weight % monomer is greater than or equal to 80% wt/wt, 83% wt/wt, 85% wt/wt or 87% wt/wt.

In some cases, the pH is from 4.0 to 7.0. In some cases, the pH is from about 4.0 to about 7.0, e.g., 4.0 to 6.0, 4.0 to 5.0, 5.0 to 7.0, 5.0 to 6.0, or 6.0 to 7.0.

In some cases, the pH is less than or equal to 5.5 (e.g., less than or equal to 5.4, 5.3, 5.2, 5.1, 5.0, 4.9, 4.8, 4.7, 4.6, 4.5, 4.4, 4.3, 4.2, 4.1, or 4.0). In some cases, the pH is from 5.2 to 5.5, e.g., 5.2 to 5.4, 5.2 to 5.3, 5.3 to 5.5, 5.3 to 5.4, or 5.4 to 5.5. In some cases, the pH is from about 5.2 to about 5.5, e.g., about 5.2 to about 5.4, about 5.2 to about 5.3, about 5.3 to about 5.5, about 5.3 to about 5.4, or about 5.4 to about 5.5. In some cases, the pH is or is about 5.3. In some cases, the pH is or is about 5.4.

In some cases, the pH is measured pre-fill. In some cases, the pH is measured after filtration. In some cases, the pH is measured at or at about one week or less after filtration.

In some cases, the pH is measured after filtration and storage at 40° C. or less, e.g., 35° C., 30° C., 25° C., 20° C., 15° C., 10° C., 5° C. or less. In some cases, the pH is measured after filtration and storage at about 40° C. or less, e.g., about 35° C., about 30° C., about 25° C., about 20° C., about 15° C., about 10° C., about 5° C., about 0° C. or less. In some cases, the pH is measured after filtration and storage at or at about -70° C.

In some cases, the pH is measured after filtration and storage at from -70° C. to 40° C., e.g., -70 to 40, -70 to 35, -70 to 30, -70 to 25, -70 to 20, -70 to 15, -70 to 10, -70 to 5, -70 to 0, 0 to 40, 0 to 35, 0 to 30, 0 to 25, 0 to 20, 0 to 15, 0 to 10, 0 to 5, 5 to 35, 5 to 30, 5 to 25, 5 to 20, 5 to 15, 5 to 10, 10 to 40, 10 to 35, 10 to 30, 10 to 25, 10 to 20, 10 to 15, 15 to 40, 15 to 35, 15 to 30, 15 to 25, 15 to 20, 20 to 40, 20 to 35, 20 to 30, 20 to 25, 25 to 40, 25 to 35, 25 to 30, 30 to 40, 30 to 35, or 35 to 40° C. In some cases, the pH is measured after filtration and storage at from about -70° C. to about 40° C., e.g., about -70 to about 40, about -70 to about 35, about -70 to about 30, about -70 to about 25, about -70 to about 20, about -70 to about 15, about -70 to about 10, about -70 to about 5, about -70 to about 0, about 0 to about 40, about 0 to about 35, about 0 to about 30, about 0 to about 25, about 0 to about 20, about 0 to about 15, about 0 to about 10, about 0 to about 5, about 5 to about 35, about 5 to about 30, about 5 to about 25, about 5 to about 20, about 5 to about 15, about 5 to about 10, about 10 to about 40, about 10 to about 35, about 10 to about 30, about 10 to about 25, about 10 to about 20, about 10 to about 15, about 15 to about 40, about 15 to about 35, about 15 to about 30, about 15 to about 25, about 15 to about 20, about 20 to about 40, about 20 to about 35, about 20 to about 30, about 20 to about 25, about 25 to about 40, about 25 to about 35, about 25 to about 30, about 30 to about 40, about 30 to about 35, or about 35 to about 40° C.

In some cases, the pH of the pharmaceutical compositions is modulated such that the weight % of monomer is altered. In some cases, the pH of the pharmaceutical compositions is lowered such that the weight % monomer is increased.

In some cases, pH of the pharmaceutical compositions is increased such that the weight % dimer is increased.

In some cases, the pH is such that the monomer weight % is greater than or equal to 85% wt/wt (e.g., greater than or equal to 86, 97, 88, 89, 90, 91, 92, 93, 94, 95, 96, 97, 98, or 99% wt/wt).

In some cases, the composition is a high concentration hsIgG composition. In some cases, the concentration of hsIgG in the composition is from 100 mg/mL to 275 mg/mL, e.g., 100 to 250, 100 to 200, 100 to 175, 100 to 125, 125 to 275, 125 to 250, 125 to 200, 125 to 175, 175 to 275, 175 to 250, 175 to 200, 200 to 275, 200 to 250, or 250 to 275 mg/mL. In some cases, the concentration of hsIgG in the composition is from about 100 mg/mL to about 275 mg/mL, e.g., about 100 to about 250, about 100 to about 200, about 100 to about 175, about 100 to about 125, about 125 to about 275, about 125 to about 250, about 125 to about 200, about 125 to about 175, about 175 to about 275, about 175 to about 250, about 175 to about 200, about 200 to about 275, about 200 to about 250, or about 250 to about 275 mg/mL. In some cases, the concentration of hsIgG in the concentration is or is about 100, 125, 175, 200, 250, or 275 mg/mL.

In some cases, the composition comprises one or more buffers. In some cases, the buffer is selected from the group consisting of sodium acetate, histidine, sodium phosphate, and combinations thereof.

In some cases, the composition comprises a tonicity modifier. In some cases, the tonicity modifier is selected from the group consisting of glycine, sorbitol, and combinations thereof.

In some cases, the composition comprises a surfactant. In some cases, the surfactant is polysorbate. In some cases, the polysorbate is polysorbate 20.

In some cases, the composition comprises both a buffer and a tonicity modifier. In some cases, the composition comprises a buffer, a tonicity modifier, and polysorbate (e.g., polysorbate 20).

In some cases, the composition comprises a sodium acetate buffer and a sorbitol tonicity modifier. In some cases, the composition comprises a sodium acetate buffer, a sorbitol tonicity modifier, and polysorbate (e.g., polysorbate 20).

In some cases, the buffer is present in the composition at 5 to 20 mM, e.g., 5 to 15, 5 to 10, 10 to 20, 10 to 15, or 15 to 20 mM. In some cases, the buffer is present in the composition at about 5 to about 20 mM, e.g., about 5 to about 15, about 5 to about 10, about 10 to about 20, about 10 to about 15, or about 15 to about 20 mM. In some cases the buffer is present in the composition at or at about 10 mM.

In some cases, the polysorbate 20 is present in the composition at 0.01% to 0.03%, e.g., 0.01% to 0.02%, 0.02% to 0.03%. In some cases, the polysorbate 20 is present in the composition at about 0.01% to about 0.03%, e.g., about 0.01% to about 0.02%, about 0.02% to about 0.03%. In some cases, the polysorbate 20 is present in the composition at or at about 0.02%.

In some cases, the glycine is present in the composition at 200 to 300 mM, e.g., 225 to 300, 225 to 275, 225 to 250, 250 to 300, 250 to 275, or 275 to 300 mM. In some cases the glycine is present in the composition at about 200 to about 300 mM, e.g., about 225 to about 300, about 225 to about 275, about 225 to about 250, about 250 to about 300, about 250 to about 275, or about 275 to about 300 mM. In some embodiments, the glycine is present in the composition at or at about 250 mM.

In some cases, the sorbitol is present in the composition at 1% to 10% (w/v), e.g., 1 to 9, 1 to 8, 1 to 7, 1 to 6, 1 to 5, 1 to 4, 1 to 3, 1 to 2, 2 to 10, 2 to 9, 2 to 8, 2 to 7, 2 to 6, 2 to 5, 2 to 4, 2 to 3, 3 to 10, 3 to 9, 3 to 8, 3 to 7, 3 to 6, 3 to 5, 3 to 4, 4 to 10, 4 to 9, 4 to 8, 4 to 7, 4 to 6, 4 to 5, 5 to 10, 5 to 9, 5 to 8, 5 to 7, 5 to 6, 6 to 10, 6 to 9, 6 to 8, 6 to 7, 7 to 10, 7 to 9, 7 to 8, 8 to 10, 8 to 9, or 9 to 10 % (w/v). In some cases, the sorbitol is present in the composition at about 1% to about 10% (w/v), e.g., about 1 to about 9, about 1 to about 8, about 1 to about 7, about 1 to about 6, about 1 to about 5, about 1 to about 4, about 1 to about 3, about 1 to about 2, about 2 to about 10, about 2 to about 9, about 2 to about 8, about 2 to about 7, about 2 to about 6, about 2 to about 5, about 2 to about 4, about 2 to about 3, about 3 to about 10, about 3 to about 9, about 3 to about 8, about 3 to about 7, about 3 to about 6, about 3 to about 5, about 3 to about 4, about 4 to about 10, about 4 to about 9, about 4 to about 8, about 4 to about 7, about 4 to about 6, about 4 to about 5, about 5 to about 10, about 5 to about 9, about 5 to about 8, about 5 to about 7, about 5 to about 6, about 6 to about 10, about 6 to about 9, about 6 to about 8, about 6 to about 7, about 7 to about 10, about 7 to about 9, about 7 to about 8, about 8 to about 10, about 8 to about 9, or about 9 to about 10 % (w/v). In some cases, the sorbitol is present in the composition at or at about 5%.

### Storage of Immunoglobulin Compositions

In some cases, the hsIgG compositions described herein are stored under various conditions prior to administration.

In some cases, the hsIgG compositions described herein retain disialylation during storage.

Thus, in some cases, in the hsIgG compositions described herein at least 60%, 70%, 80%, 90% or 95% of branched glycans on the Fc domain of the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22, or 24 months at -70° C., 4° C., 5° C., 25° C., or 40° C.

In some cases, at least 60%, 70%, 80%, 90% or 95% of branched glycans on the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C.

In some cases, at least 60%, 70%, 80%, 90% or 95% of branched glycans on the Fab domain of the immunoglobulins are disialylated by way of NeuAc-α 2,6-Gal terminal linkages after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C.

In some cases, 5-10% of the immunoglobulins are dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C.

In some cases, at least 85% of the immunoglobulins are monomers or dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C.

In some cases, at least 90% of the immunoglobulins are monomers or dimers after storage for 1, 2, 3, 4, 6, 8, 10, 12, 14, 16, 18, 20, 22, or 24 weeks or 1, 2, 3, 4, 6, 8, 10, 12, 14, 16, 18, 20, 22 or 24 months at -70° C., 4° C., 5° C., 25° C., or 40° C.

In some cases, the composition is stable at 5° C. for at least 7 months, at 25° C. for at least one month, 2-8° C. for two years, and/or two weeks at 15-30° C.

In some cases, the compositions described herein are stored in a sealed US Pharmacopeia Type 1 glass vial. In some cases, the hsIgG compositions described herein are stored in a sealed 2R Type 1 glass injection vial.

In some cases, the compositions described herein are provided in a pre-filled syringe.

### Pharmaceutical Compositions and Administration

Hypersialylated IgG can be incorporated into a pharmaceutical composition. For example, the pharmaceutical composition can be formulated by suitably combining the hsIgG with pharmaceutically acceptable vehicles or media, such as sterile water and physiological saline, vegetable oil, emulsifier, suspension agent, surfactant, stabilizer, flavoring excipient, diluent, vehicle, preservative, binder, followed by mixing in a unit dose form required for generally accepted pharmaceutical practices. The amount of active ingredient included in the pharmaceutical preparations is such that a suitable dose within the designated range is provided.

The hsIgG can be formulated for intravenous administration. The hsIgG can also be formulated for subcutaneous administration.

The pharmaceutical composition can be formulated in accordance with conventional pharmaceutical practices using distilled water for injection as a vehicle. For example, physiological saline or an isotonic solution containing glucose and other supplements such as D-sorbitol, D-mannose, D-mannitol, and sodium chloride may be used as an aqueous solution for injection, optionally in combination with a suitable solubilizing agent, for example, alcohol such as ethanol and polyalcohol such as propylene glycol or polyethylene glycol, and a nonionic surfactant such as polysorbate 80™, HCO-50 and the like.

Nonlimiting examples of oily liquid include sesame oil and soybean oil, and it may be combined with benzyl benzoate or benzyl alcohol as a solubilizing agent. Other items that may be included are a buffer such as a phosphate buffer, or sodium acetate buffer, a soothing agent such as procaine hydrochloride, a stabilizer such as benzyl alcohol or phenol, and an antioxidant. The formulated injection can be packaged in a suitable container, e.g., as described herein.

A suitable means of administration can be selected based on the age and condition of the patient. A suitable dose of hsIgG prepared by the methods described herein can be about the same or less than (e.g., 20%, 35%, 40%, 50%, 60%, 70% or 80% less) the suitable or approved dose of commercially available IVIg preparations. The dose and method of administration varies depending on the weight, age, condition, and the like of the patient, and can be suitably selected as needed by those skilled in the art.

In some embodiments, the dose is 1% - 10% of the FDA approved (or other national or international regulatory agency) IVIG dose or the effective IVIG dose for a disorder. In some embodiments, the FDA (or other national or international regulatory agency) approved dose or effective dose of IVIG is 200 mg/kg, 400 mg/kg, 500 mg/kg, 600 mg/kg, 1000 mg/kg, or 2000 mg/kg. In some embodiments, a composition comprising a hsIgG preparation is administered at a dose of about 4, 5, 6, 10, 15, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 975, or 1000 mg/kg. In some embodiments, a composition comprising a hsIgG preparation is administered daily, weekly, semiweekly, biweekly, monthly, semimonthly, bimonthly, every 3 days, every 4 days, every 5 days, every 6 days, every 7 days, once every 14 days, once every 21 days, once every 28 days, once daily for two consecutive days in a 28-day cycle, or with the same administration frequency as the FDA approved IVIG dose. In some embodiments, a composition is administered in a single dose. In some embodiments, a composition is administered in multiple doses. The dose and method of administration varies depending on the weight, age, condition, and the like of the patient, and can be suitably selected as needed by those skilled in the art.

As described in Washburn et al., analysis of sialylation by ST6Gal1 revealed that

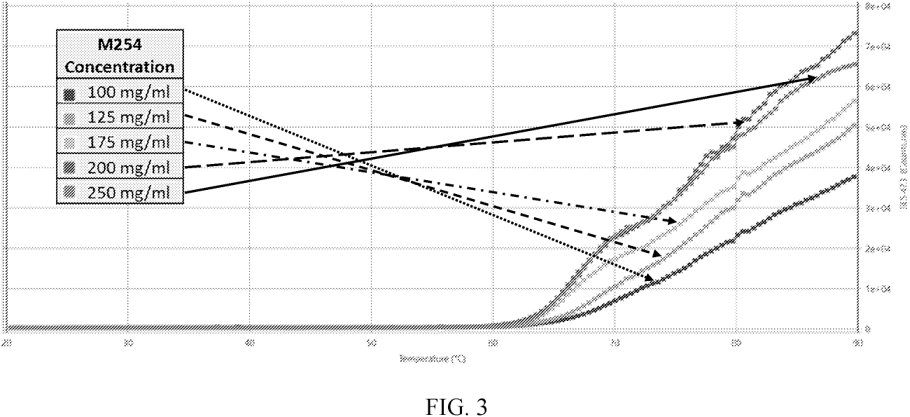

ST6Gal1 can not only catalyze the transfer of the sialic acid from CMP-NANA sugar nucleotide to the Fc glycan but also, facilitate the removal of the sialic acid from the sialylated product. The reactions are shown schematically in FIG. 3.

As Washburn et al. reported, under certain reaction conditions, sialylation of the α1,3 branch of the biantennary glycan (to form A1F-1,3) was rapid and essentially complete by 30 min, whereas the doubly sialylated species (A2F) formed at an ~ 10× slower rate and stopped accumulating by 24 h. After 24 h, a monosialylated species with the sialic acid on the α1,6 branch(A1F-1,6) started to form and continued accumulating steadily, reaching ~35% of glycosylated species by 64 h. Concomitantly, A2F exhibited a steady decline from 71% at 20 h to 44% at 64 h, suggesting that the A1F-1,6 glycoform was generated from the removal of sialic acid residues on the more exposed α1,3 branch of disialylated A2F. Additional incubation led to the cleavage of the 1,6 sialic acid in the A1F-1,6 glycoform, generating the asialylated but fully galactosylated and fucosylated species G2F. G2F, which was present in trace amounts at the beginning of the reaction, appeared at measurable amounts at 40 h and continued increasing, reaching 15% by 64 h as the levels of A2F and A1F-1,6 declined.

Washburn et al. further reported that these observations were critical in optimizing the parameters to maximize the yield of the A2F species while minimizing the

A1F-1,3, A1F-1,6, and G2F glycoforms. By evaluating a matrix of parameters affecting the transient state of the G2F⇌ A1F-1,3⇌A2F⇌A1F-1,6⇌G2F glycoform distribution, they found that the desialylation component of the reaction (A2F⇌A1F-1,6⇌G2F) was facilitated by spontaneous decomposition of CMP-NANA in the reaction. Wasburn et al. concluded that replenishing the CMP-NANA in the sialylation reaction assists in maximize the A2F yield and found that periodic dosing of fresh CMP-NANA maximized the A2F glycan levels after 24 h without the formation of substantial A1F-1,6 or G2F species.

### EXAMPLES

The invention is further described in the following examples, which do not limit the scope of the invention described in the claims.

### Example 1: Hypersialylated IgG Formulated in Different Buffers

Hypersialylated IgG in which more than 60% of the branched Fc region glycans are disialylated was prepared as generally described in WO2014/179601.

Briefly, IVIg is exposed to a one-pot sequential enzymatic reaction using β1,4 galactosyltransferase 1 (B4-GalT) and α2,6-sialyltransferase (ST6-Gal1) enzymes. The galactosyltransferase enzyme selectively adds galactose residues to pre-existing asparagine-linked glycans in IVIg. The resulting galactosylated glycans serve as substrates to the sialic acid transferase enzyme which selectively adds sialic acid residues to cap the asparagine-linked glycan structures attached to IVIg. Thus, the overall sialylation reaction employed two sugar nucleotides (UDPGal and CMP-NANA). The latter was replenished periodically to increase di-sialylated product relative to monosialylated product. The reaction includes the co-factor manganese chloride.

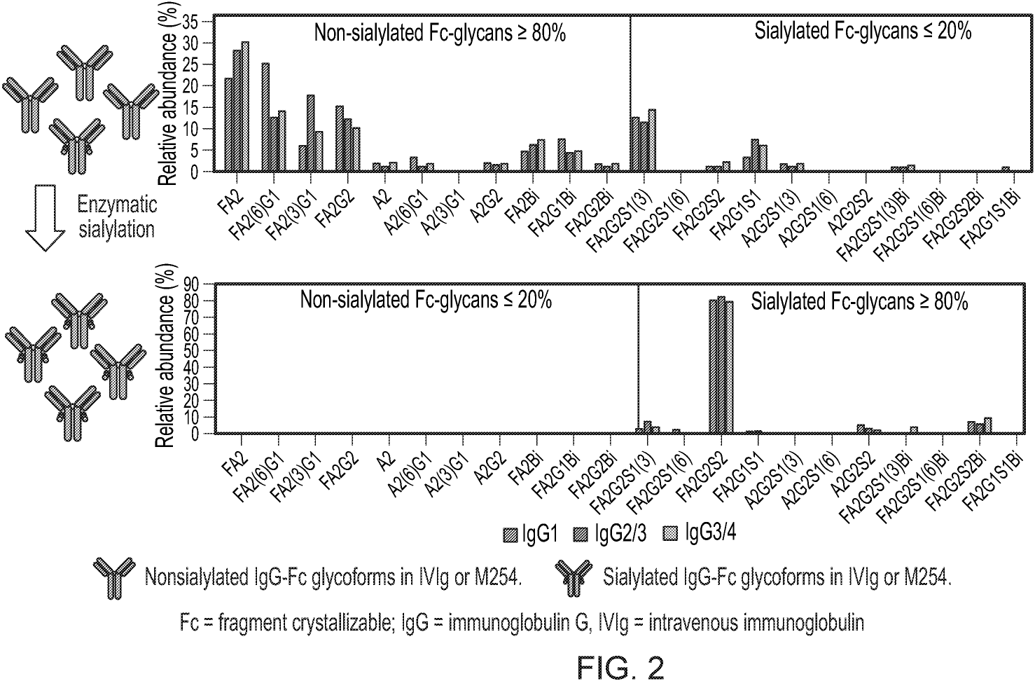

A representative example of the corresponding IgG-Fc glycan profile for the starting IVIg and the reaction product is shown in the right panel of FIG. 2. The glycan data is shown per IgG subclass. Glycans from IgG3 and IgG4 subclasses cannot be quantified separately. As shown, for IVIg the sum of all the nonsialylated glycans is more than 80% and the sum of all sialylated glycans is < 20%. For the reaction product, the sum for all nonsialylated glycans is < 20% and the sum for all sialylated glycans is more than 80%. Nomenclature for different glycans listed in the glycoprofile use the Oxford notation for N linked glycans. FIG. 2 shows a schematic representation of enzymatic sialylation reaction to transform IVIg to hsIgG (left); IgG Fc glycan profile for the starting IVIg and hsIgG. Glycan profiles for the different IgG subclasses are derived via glycopeptide mass spectrometry analysis. Peptide sequences used to quantify glycopeptides for different IgG subclasses are: IgG1 = EEQYNSTYR (SEQ ID NO:1), IgG2/3 EEQFNSTFR (SEQ ID NO:2), IgG3/4 EEQYNSTFR (SEQ ID NO:3) and EEQFNSTYR (SEQ ID NO:4) (right).

IVIg that is not hypersialylated, including commercially available IVIg, is generally stable in glycine and generally does not form subvisible particles when agitated, for example, during shipping.

### Example 2: Various hsIgG Formulations

Stability assays were conducted to assess a range of liquid formulations to determine the best suited conditions providing maximum stability of M254 (hypersialylated immunoglobulin) at high concentrations. Formulation differences included buffer, pH, and tonicity modifier while concentration and surfactant percentages were kept consistent throughout all samples. Then, various stresses were induced that are related to pharmaceutical processing, storage, and shipping.

Formulations composed with sodium acetate buffer at low pH were consistently stable at various temperatures and over time. In particular, the sodium acetate buffer and sorbitol tonicity modifier combination exhibited increased stability compared to the sodium acetate buffer and glycine combination. Histidine buffers and sodium phosphate buffers were higher in pH and were not as stable, exhibiting variability compared to the sodium acetate buffer.

Fourteen different formulations (F1 to F14) ranging from pH 4.2 to 7.5 at 100 mg/mL were prepared for the studies described herein as shown in Table 1.

TABLE 1

Different hsIgG formulations (F1 to F14) ranging from pH 4.2 to 7.5 at 100 mg/mL

| Form. No. | Buffer (10 mM) | pH | Tonicity Modifier | API (mg/mL) | Surfactant |
|---|---|---|---|---|---|
| 1 | Sodium Acetate | 4.2 | 250 mM Glycine | 100 | 0.02% PS20 |
| 2 | Sodium Acetate | 4.7 | 5% Sorbitol | 100 | 0.02% PS20 |
| 3 | Sodium Acetate | 5.0 | 250 mM Glycine | 100 | 0.02% PS20 |
| 4 | Sodium Acetate | 5.0 | 5% Sorbitol | 100 | 0.02% PS20 |
| 5 | Histidine | 6.0 | 250 mM Glycine | 100 | 0.02% PS20 |
| 6 | Histidine | 6.0 | 5% Sorbitol | 100 | 0.02% PS20 |
| 7 | Sodium Phosphate | 6.0 | 250 mM Glycine | 100 | 0.02% PS20 |
| 8 | Sodium Phosphate | 6.0 | 5% Sorbitol | 100 | 0.02% PS20 |
| 9 | Sodium Phosphate | 7.0 | 250 mM Glycine | 100 | 0.02% PS20 |
| 10 | Sodium Phosphate | 7.0 | 5% Sorbitol | 100 | 0.02% PS20 |
| 11 | Sodium Phosphate | 7.5 | 250 mM Glycine | 100 | 0.02% PS20 |
| 12 | Sodium Phosphate | 7.5 | 5% Sorbitol | 100 | 0.02% PS20 |
| 13 | None | 5.2 | Glycine | 100 | None |
| 14 | None | 5.2 | Glycine | 100 | 0.02% PS20 |

### Example 3: Stabilized Glycine Formulation of hsIgG With Different Buffers

Samples formulations post-filtration were transferred to glass vials and exposed to four stress temperatures (-70° C., 5° C., 25° C., or 40° C.) over twelve weeks. Following exposure to stress temperatures, comparable concentration and pH were observed compared to their corresponding unstressed condition for every formulation. The results are shown in Table 2.

The sodium acetate buffer formulations (F1, F3) were clear of visible particles as were the controls (F13, F14). All other buffer formulations (F5, F7, F9, and F11) exhibited increasing opalescence with increasing pH at all temperatures. All formulations at 40° C. exhibited yellow coloration with increasing pH but were free of visible particles.

TABLE 2

Stability of hsIgG in 250 mM glycine/0.02% with various 10 mM buffers after 12 weeks of storage

| Form No. | Buffer | pH | T = 12 Weeks | | | |
|---|---|---|---|---|---|---|
| | | | -70 OC | 5 OC | 25 OC | 40 OC |
| F1 | Sodium Acetate | 4.2 | Clear, no particles | Clear, no particles | Clear, no particles | Clear, no particles |
| F3 | Sodium Acetate | 5 | Clear, no particles | Clear, no particles | Clear, no particles | Clear, no particles |
| F5 | Histidine | 6 | Opalescence | Opalescence | Opalescence | Opalescence |
| F7 | Sodium Phosphate | 6 | Opalescence | Opalescence | Opalescence | Opalescence |
| F9 | Sodium Phosphate | 7 | Opalescence | Opalescence | Opalescence | Opalescence |
| F11 | Sodium Phosphate | 7.5 | Opalescence | Opalescence | Opalescence | Opalescence |
| F13 | None -Control 1 | 5.2 | Clear, no particles | Clear, no particles | Clear, no particles | Clear, no particles |
| F14 | None -Control 2 | 5.2 | Clear, no particles | Clear, no particles | Clear, no particles | Clear, no particles |

### Example 4: Stabilized Glycine Formulation of hsIgG With Different Buffers at Various Temperatures

Sample formulations post filtration were transferred to glass vials and stored at stress temperatures (-70° C., 5° C., 25° C., or 40° C.) over twelve weeks. Following storage at stress temperature, formulations were tested for purity by SE-HPLC. The results are shown in Tables 3-5.

Sodium acetate buffer formulations exhibited less aggregate formation that all other formulations including the control. F3 exhibited the least amount of aggregate formation at all stress temperature ranges. F1 had minimal aggregate formation, similar to the controls at all stress temperatures except at 40° C., where there was an increase in aggregate formation.

At time zero, formulations with pH 6 and above displayed lower monomer peak percentages comparted to formulations at lower pH. Formulations at pH 5 and below exhibited the highest monomer percentages compared to other formulations, consistent with previous studies.

All formulations displayed increases in dimer peak percentages compared at time zero. Larger increases in dimer formation correlated with increase in pH, as seen in previous studies. Acetate buffer preserved the monomer and dimer percentages, with smaller dimer conversions, at the temperature stresses better than all other formulations, including the controls.

TABLE 3

Impact of Temperature Stress Over Time on Aggregate Percentage

| | | | T=0 | T = 12 weeks | | | |
|---|---|---|---|---|---|---|---|
| Form. No. | Buffer | pH | | -70C | 5C | 25C | 40C |
| | | | Aggregate (%) | Aggregate (%) | Aggregate (%) | Aggregate (%) | Aggregate (%) |
| F1 | Sodium Acetate | 4.2 | 0.4 | 0.4 | 0.4 | 0.4 | 5.9 |
| F3 | Sodium Acetate | 5 | 0.6 | 0.6 | 0.7 | 0.7 | 2.9 |
| F5 | Histidine | 6 | 0.7 | 0.9 | 1.1 | 1.1 | 3.5 |
| F7 | Sodium Phosphate | 6 | 0.6 | 0.8 | 1.1 | 1.2 | 5.1 |
| F9 | Sodium Phosphate | 7 | 0.7 | 1 | 1.5 | 1.7 | 6.1 |
| F11 | Sodium Phosphate | 7.5 | 0.7 | 1 | 1.5 | 2.3 | 9.3 |
| F13 | None -Control 1 | 5.2 | 0.6 | 0.7 | 0.7 | 0.9 | 4.5 |
| F14 | None -Control 2 | 5.2 | 0.5 | 0.7 | 0.8 | 1 | 4.7 |

TABLE 4

Impact of Temperature Stress Over Time on Monomer Stability

| | | | T=0 | T = 12 weeks | | | |
|---|---|---|---|---|---|---|---|
| Form. No. | Buffer | pH | | -70C | 5C | 25C | 40C |
| | | | Monomer (%) | Monomer (%) | Monomer (%) | Monomer (%) | Monomer (%) |
| F1 | Sodium Acetate | 4.2 | 93.6 | 92.8 | 92.1 | 93 | 88 |
| F3 | Sodium Acetate | 5 | 90.3 | 89.1 | 88.3 | 88.3 | 87.3 |
| F5 | Histidine | 6 | 88.3 | 85.9 | 83.9 | 83.5 | 84 |
| F7 | Sodium Phosphate | 6 | 88.8 | 86.2 | 84.3 | 83.8 | 80.6 |
| F9 | Sodium Phosphate | 7 | 88.1 | 84.8 | 82.2 | 81.2 | 78.6 |
| F11 | Sodium Phosphate | 7.5 | 88.3 | 84.9 | 81.8 | 80 | 75.1 |
| F13 | None -Control 1 | 5.2 | 90.6 | 88.2 | 87.7 | 87 | 83.4 |
| F14 | None -Control 2 | 5.2 | 90.5 | 88.2 | 87.7 | 87 | 83.2 |

TABLE 5

Impact of Temperature Stress over Time on Dimer Stability

| | | | T=0 | T = 12 weeks | | | |
|---|---|---|---|---|---|---|---|
| Form. No. | Buffer | pH | | -70C | 5C | 25C | 40C |
| | | | Dimer (%) | Dimer (%) | Dimer (%) | Dimer (%) | Dimer (%) |
| F1 | Sodium Acetate | 4.2 | 6 | 6.9 | 7.4 | 6.6 | 6.1 |
| F3 | Sodium Acetate | 5 | 9.1 | 10.3 | 11 | 11 | 9.8 |
| F5 | Histidine | 6 | 11 | 13.2 | 14.9 | 15.3 | 12.5 |
| F7 | Sodium Phosphate | 6 | 10.6 | 13 | 14.6 | 15 | 14.3 |
| F9 | Sodium Phosphate | 7 | 11.2 | 14.1 | 16.3 | 17.1 | 15.3 |
| F11 | Sodium Phosphate | 7.5 | 11 | 14.2 | 16.8 | 17.7 | 15.6 |
| F13 | None -Control 1 | 5.2 | 8.9 | 11.1 | 11.5 | 12.1 | 12.1 |
| F14 | None -Control 2 | 5.2 | 9 | 11.1 | 11.5 | 12.1 | 12.1 |

### Example 5: Stability of N-Glycan and % Sialylation and Disialylation in Glycine Formulation of hsIgG with Different Buffers

Sample formulations post filtration were transferred to glass vials and stored at stress temperatures (-70° C., 5° C., 25° C., or 40° C.) over twelve weeks. Following storage at stress temperature, formulations were tested for purity by HILIC-HPLC. At time zero, all formulations displayed comparable A2F peak percentages (between 68.4% to 68.8%). The results are shown in Tables 6-9.

Storage at -70° C. and 5° C. resulted in comparable A2F peak percentages (70.0% to 70.9% for the former, and 69.9% to 70.4% for the latter), total sialylation (99.5% to 99.6%), monosialylation percentage (6.7% to 7.7%), and disialylated percentage (91.4% to 92.8%).

At 25° C., F1 exhibited decreases in A2F. All other formulations at pH 5.0 and above displayed slight increases in peak A2F.

At 40° C., F1 exhibited significant changes in A2F, most likely due to the lower pH, whereas higher pH formulations (>pH 6.5, F9, F11) showed the least amount of change in N-glycan peak percentages.

F3, sodium acetate buffer, exhibited A2F peaks, percent total sialylation, percent monosialylation, and percent disialylation similar to that of the controls for all stress temperatures. F1, also a sodium acetate buffer, exhibited the most variability compared to all other formulations.

TABLE 6

A2F Glycan Stability at Different Temperatures

| | | | T=0 | T = 12 weeks | | | |
|---|---|---|---|---|---|---|---|
| Form. No. | Buffer | pH | | -70C | 5C | 25C | 40C |
| | | | A2F | A2F | A2F | A2F | A2F |
| F1 | Sodium Acetate | 4.2 | 68.8 | 70.2 | 70 | 64.9 | 36.5 |
| F3 | Sodium Acetate | 5 | 68.6 | 70.3 | 70.1 | 69.3 | 60.8 |
| F5 | Histidine | 6 | 68.7 | 70.4 | 70.3 | 70.1 | 67.3 |
| F7 | Sodium Phosphate | 6 | 68.7 | 70.2 | 70.2 | 69.7 | 67.3 |
| F9 | Sodium Phosphate | 7 | 68.5 | 70.2 | 70.3 | 69.7 | 69.1 |
| F11 | Sodium Phosphate | 7.5 | 68.7 | 70.7 | 70.1 | 70.4 | 69.6 |
| F13 | None - Control 1 | 5.2 | 68.6 | 70.4 | 70.4 | 69.3 | 62.7 |
| F14 | None - Control 2 | 5.2 | 68.8 | 70 | 70.1 | 69.2 | 62.7 |

TABLE 7

Total Percent Sialylation at Different Temperatures

| | | | T=0 | T = 12 weeks | | | |
|---|---|---|---|---|---|---|---|
| | | | | -70C | 5C | 25C | 40C |
| Form. No | Buffer | pH | Total Sialylation (% Area) | Total Sialylation (% Area) | Total Sialylation (% Area) | Total Sialylation (% Area) | Total Sialylation (% Area) |
| F1 | Sodium Acetate | 4.2 | 99.5 | 99.6 | 99.6 | 99.2 | 93 |
| F3 | Sodium Acetate | 5 | 99.5 | 99.5 | 99.6 | 99.6 | 99.1 |
| F5 | Histidine | 6 | 99.4 | 99.6 | 99.6 | 99.6 | 99.6 |
| F7 | Sodium Phosphate | 6 | 99.5 | 99.6 | 99.6 | 99.5 | 99.5 |
| F9 | Sodium Phosphate | 7 | 99.5 | 99.5 | 99.6 | 99.6 | 99.5 |
| F11 | Sodium Phosphate | 7.5 | 99.5 | 99.5 | 99.6 | 99.6 | 99.6 |
| F13 | None -Control 1 | 5.2 | 99.5 | 99.6 | 99.6 | 99.6 | 99.2 |
| F14 | None -Control 2 | 5.2 | 99.5 | 99.6 | 99.6 | 99.5 | 99.2 |

TABLE 8

Monosialylation at Different Temperatures

| | | | T=0 | T = 12 weeks | | | |
|---|---|---|---|---|---|---|---|
| | | | | -70C | 5C | 25C | 40C |
| Form. No. | Buffer | pH | Mono-sialylated | Mono-sialylated | Mono-sialylated | Mono-sialylated | Mono-sialylated |
| | | | (% Area) | (% Area) | (% Area) | (% Area) | (% Area) |
| F1 | Sodium Acetate | 4.2 | 7.8 | 7.4 | 7.6 | 14.3 | 46.3 |
| F3 | Sodium Acetate | 5 | 8 | 7.2 | 7.7 | 8.4 | 19.2 |
| F5 | Histidine | 6 | 7.9 | 7 | 7.4 | 7.4 | 10.7 |
| F7 | Sodium Phosphate | 6 | 7.9 | 7.2 | 7.4 | 7.9 | 10.9 |
| F9 | Sodium Phosphate | 7 | 7.9 | 7.3 | 7.3 | 7.5 | 8.1 |
| F11 | Sodium Phosphate | 7.5 | 7.9 | 6.7 | 7.5 | 6.9 | 7.8 |
| F13 | None -Control 1 | 5.2 | 8 | 7.3 | 7 | 8.4 | 16.7 |
| F14 | None -Control 2 | 5.2 | 7.9 | 7.3 | 7.5 | 8.3 | 16.9 |

TABLE 9

Disialylation at Different Temperatures

| | | | T=0 | T = 12 weeks | | | |
|---|---|---|---|---|---|---|---|
| | | | Time Zero | -70C | 5C | 25C | 40C |
| Form. No. | Buffer | pH | Disialylated (% Area) | Disialylated (% Area) | Disialylated (% Area) | Disialylated (% Area) | Disialylated (% Area) |
| F1 | Sodium Acetate | 4.2 | 91.5 | 92.2 | 91.9 | 84.9 | 46.7 |
| F3 | Sodium Acetate | 5 | 91.5 | 92.4 | 91.9 | 91.1 | 79.9 |
| F5 | Histidine | 6 | 91.5 | 92.6 | 92.2 | 92.2 | 88.9 |
| F7 | Sodium Phosphate | 6 | 91.5 | 92.3 | 92.2 | 91.6 | 88.7 |
| F9 | Sodium Phosphate | 7 | 91.5 | 92.3 | 92.3 | 92.1 | 91.4 |
| F11 | Sodium Phosphate | 7.5 | 91.4 | 92.8 | 92.1 | 92.7 | 91.8 |
| F13 | None -Control 1 | 5.2 | 91.5 | 92.3 | 92.5 | 91.1 | 82.5 |
| Form. No. | Buffer | pH | Disialylated (% Area) | Disialylated (% Area) | Disialylated (% Area) | Disialylated (% Area) | Disialylated (% Area) |
| F14 | None -Control 2 | 5.2 | 91.5 | 92.2 | 92.1 | 91.2 | 82.3 |

### Example 6: Charge Variants Within Stabilized Glycine Formulation of hsIgG with Various Buffers at Various Stress Temperatures

Sample formulations post filtration were transferred to glass vials, and stored at stress temperatures (-70° C., 5° C., 25° C., or 40° C.) over twelve weeks. Following storage at stress temperature, formulations were tested for changes in charge-variants and isoelectric peaks by imaged capillary isoelectric focusing (icIEF) to understand formulation stability at different stress temperatures. At time zero, all formulations exhibited similar acid, neutral and basic combined peak percentages, as well as a main peak isoelectric point near 7.4. All main peak isoelectric points remained around 7.4 regardless of temperature stress. The results are shown in Tables 10-13.

Formulations exhibited negligible changes in isoform percentages with no significant trends observed at -70° C., 5° C., and

At 25° C., all formulations (except F14) displayed slight decreases in basic peak percentages (-0.15 to 3.41%), with corresponding increases across neutral and acidic percentages.

At 40° C. F3, sodium acetate buffer formulation, showed comparable acidic, neutral and basic peak percentages to the control formulations with negligible changes from time zero. All formulations showed decreases in basic peak percentages (except F1) and increases in acidic peak percentages (except F1), and neutral peak percentages (except F11). F11, a sodium phosphate buffer, showed the larges shifts in peak percentages since time zero.

TABLE 10

Acidic Charge Variant changes over Time and Temperature Stresses

| | | T=0 | T = 12 weeks | | | |
|---|---|---|---|---|---|---|
| | | Time Zero | -70C | 5C | 25C | 40C |
| Form. No. | Buffer | Acidic (%) | Acidic (%) | Acidic (%) | Acidic (%) | Acidic (%) |
| F1 | Sodium Acetate | 23.65 | 24.08 | 24.07 | 23.59 | 17.98 |
| F3 | Sodium Acetate | 23.18 | 24.2 | 24.09 | 24.11 | 24.98 |
| F5 | Histidine | 23.48 | 24.3 | 24.15 | 24.34 | 28.04 |
| F7 | Sodium Phosphate | 23.78 | 24.23 | 24.41 | 24.18 | 27.32 |
| F9 | Sodium Phosphate | 23.99 | 24.65 | 24.29 | 25.52 | 31.01 |
| F11 | Sodium Phosphate | 23.67 | 24.12 | 24.81 | 26.69 | 38.85 |
| F13 | None | 23.5 | 24.36 | 23.64 | 24.34 | 25.51 |
| F14 | None | 23.74 | 23.56 | 23.51 | 23.78 | 25.61 |

TABLE 11

Neutral Charge Variant Changes over Time and Temperature Stresses

| | | T=0 | T = 12 weeks | | | |
|---|---|---|---|---|---|---|
| | | Time Zero | -70C | 5C | 25C | 40C |
| Form. No. | Buffer | Neutral (%) | Neutral (%) | Neutral (%) | Neutral (%) | Neutral (%) |
| F1 | Sodium Acetate | 31.71 | 32.48 | 31.74 | 32.19 | 36.75 |
| F3 | Sodium Acetate | 32.3 | 32.68 | 32.43 | 31.89 | 34.01 |
| F5 | Histidine | 32.63 | 33.78 | 33.02 | 32.79 | 34.39 |
| F7 | Sodium Phosphate | 32.82 | 33.5 | 33.27 | 32.57 | 34.24 |
| F9 | Sodium Phosphate | 33.02 | 33.36 | 33.6 | 33.41 | 33.11 |
| F11 | Sodium Phosphate | 32.66 | 33.2 | 33.57 | 33.06 | 30.02 |
| F13 | None | 32.42 | 32.8 | 32.5 | 32.01 | 33.7 |
| F14 | None | 32.64 | 32.19 | 32.48 | 32.15 | 33.98 |

TABLE 12

Basic Charge Variant changes over Time and Temperature Stresses

| | | T=0 | T = 12 weeks | | | |
|---|---|---|---|---|---|---|
| | | | -70C | 5C | 25C | 40C |
| Form. No. | Buffer | Basic (%) | Basic (%) | Basic (%) | Basic (%) | Basic (%) |
| F1 | Sodium Acetate | 44.65 | 43.44 | 44.19 | 44.22 | 45.27 |
| F3 | Sodium Acetate | 44.52 | 43.13 | 43.48 | 44 | 41.02 |
| F5 | Histidine | 43.88 | 41.92 | 42.83 | 42.87 | 37.57 |
| F7 | Sodium Phosphate | 43.4 | 42.27 | 42.32 | 43.25 | 38.44 |
| F9 | Sodium Phosphate | 42.99 | 41.99 | 42.11 | 41.07 | 35.89 |
| F11 | Sodium Phosphate | 43.67 | 42.67 | 41.62 | 40.26 | 31.13 |
| F13 | None | 44.07 | 42.84 | 43.86 | 43.66 | 40.78 |
| F14 | None | 43.62 | 44.25 | 44.01 | 44.07 | 40.4 |

TABLE 13

Main peak Isoelectric changes over Time and Temperature Stresses

| | | T=0 | T = 12 weeks | | | |
|---|---|---|---|---|---|---|
| | | | -70C | 5C | 25C | 40C |
| Form. No. | Buffer | Main Peak (pI) | Main Peak (pI) | Main Peak (pI) | Main Peak (pI) | Main Peak (pI) |
| F1 | Sodium Acetate | 7.4 | 7.42 | 7.42 | 7.41 | 7.42 |
| F3 | Sodium Acetate | 7.39 | 7.42 | 7.42 | 7.42 | 7.42 |
| F5 | Histidine | 7.39 | 7.42 | 7.42 | 7.41 | 7.42 |
| F7 | Sodium Phosphate | 7.4 | 7.42 | 7.42 | 7.42 | 7.42 |
| F9 | Sodium Phosphate | 7.4 | 7.42 | 7.42 | 7.42 | 7.41 |
| F11 | Sodium Phosphate | 7.4 | 7.42 | 7.42 | 7.42 | 7.41 |
| F13 | None | 7.39 | 7.42 | 7.42 | 7.41 | 7.41 |
| F14 | None | 7.4 | 7.42 | 7.42 | 7.42 | 7.41 |

### Example 7: Stabilized Sorbitol Formulation of hsIgG

Samples formulations post filtration were transferred to glass vials, and exposed to stress temperatures (-70° C., 5° C., 25° C., or 40° C.) over twelve weeks. Following exposure to stress temperatures, comparable concentration and pH were observed compared to their corresponding unstressed condition for every formulation. The results are shown in Table 14.

The sodium acetate buffer formulations (F2, F4) were clear of visible particles as were the controls (F13, F14). All other buffer formulations (F6, F8, F10, and F12) exhibited increasing opalescence with increasing pH at all temperatures. All formulations at 40° C. exhibited yellow coloration with increasing pH but were free of visible particles.

TABLE 14

Stability of hsIgG in 5% sorbitol/0.02% with various 10 mM buffers after 12 weeks of storage

| Form. No. | Buffer | pH | T= 12 Weeks | | | |
|---|---|---|---|---|---|---|
| | | | -70OC | 5OC | 25OC | 40OC |
| F2 | Sodium Acetate | 4.7 | Clear, no particles | Clear, no particles | Clear, no particles | Clear, no particles |
| F4 | Sodium Acetate | 5 | Clear, no particles | Clear, no particles | Clear, no particles | Clear, no particles |
| F6 | Histidine | 6 | Opalescence | Opalescence | Opalescence | Opalescence |
| F8 | Sodium Phosphate | 6 | Opalescence | Opalescence | Opalescence | Opalescence |
| F10 | Sodium Phosphate | 7 | Opalescence | Opalescence | Opalescence | Opalescence |
| F12 | Sodium Phosphate | 7.5 | Opalescence | Opalescence | Opalescence | Opalescence |
| F13 | None -Control 1 | 5.2 | Clear, no particles | Clear, no particles | Clear, no particles | Clear, no particles |
| F14 | None -Control 2 | 5.2 | Clear, no particles | Clear, no particles | Clear, no particles | Clear, no particles |

### Example 8: Stabilized Sorbitol Formulation of hsIgG With Various Buffers at Various Temperatures

Sample formulations post filtration were transferred to glass vials, and stored at stress temperatures (-70° C., 5° C., 25° C., or 40° C.) over twelve weeks. Following storage at stress temperature, formulations were tested for purity by SE-HPLC. The results are shown in Tables 15-17.

Sodium acetate buffer formulations exhibited less aggregate formation than all other formulations including the controls. All sorbitol formulations exhibited decrease in aggregate formation compared to previous studies with 250 mM glycine, with sodium acetate buffers with 5% sorbitol exhibiting the least amount of aggregate formations.

After 12 weeks, formulations at lower pH with sorbitol show the least amount of aggregation and dimerization (F2, F4).

At -70° C., 5° C., 25° C., formulations at pH 6 and above began to exhibit slight increase in aggregate percentages compared to time zero results. All formulations, except F2 at 40° C., displayed increases in dimer percentages compared to time zero.

After 12 weeks F2 and F4 showed the least change in dimer percentage, while control formulations exhibited increases of 3.1% to 3.2%. Further F2 and F4 showed low aggregation percentages (+1.2% to 1.7%), while the controls exhibited more aggregation (3.9% to 4.2%). At each pH, formulations with sorbitol exhibited less aggregation that formulations with glycine. Overall, formulations at lower pH with sorbitol (F2 and F4) show the least change in monomer percentage (-1.3% and -1.8%) compared to time zero, while all other formulations show decreases in monomer greater than 3.0%.

TABLE 15

Impact of Temperature Stress Over Time on Aggregate Percentage with 5% Sorbitol

| | | | T=0 | T = 12 weeks | | | |
|---|---|---|---|---|---|---|---|
| Form. No. | Buffer | pH | | -70C | 5C | 25C | 40C |
| | | | Aggregate (%) | Aggregate (%) | Aggregate (%) | Aggregate (%) | Aggregate (%) |
| F2 | Sodium Acetate | 4.7 | 0.4 | 0.5 | 0.5 | 0.6 | 2.1 |
| F4 | Sodium Acetate | 5 | 0.6 | 0.7 | 0.8 | 0.8 | 1.8 |
| F6 | Histidine | 6 | 0.6 | 0.8 | 1.1 | 1.2 | 2.7 |
| F8 | Sodium Phosphate | 6 | 0.6 | 0.8 | 1 | 1.2 | 2.3 |
| F10 | Sodium Phosphate | 7 | 0.7 | 1.1 | 1.5 | 1.7 | 3.7 |
| F12 | Sodium Phosphate | 7.5 | 0.8 | 1 | 1.6 | 2.1 | 5.3 |
| F13 | None - Control 1 (Glycine) | 5.2 | 0.6 | 0.7 | 0.7 | 0.9 | 4.5 |
| F14 | None - Control 2 (Glycine) | 5.2 | 0.5 | 0.7 | 0.8 | 1 | 4.7 |

TABLE 16

Impact of Temperature Stress over Time on monomer stability with 5% Sorbitol

| | | | T=0 | T = 12 weeks | | | |
|---|---|---|---|---|---|---|---|
| Form. No. | Buffer | pH | | -70C | 5C | 25C | 40C |
| | | | Monomer (%) | Monomer (%) | Monomer (%) | Monomer (%) | Monomer (%) |
| F2 | Sodium Acetate | 4.7 | 91.6 | 90.6 | 90.1 | 90.5 | 90.3 |
| F4 | Sodium Acetate | 5 | 90 | 88.7 | 87.7 | 87.7 | 88.2 |
| F6 | Histidine | 6 | 88.4 | 85.8 | 83.8 | 83.2 | 84.2 |
| F8 | Sodium Phosphate | 6 | 89.2 | 86.6 | 85.1 | 84.5 | 85.5 |
| F10 | Sodium Phosphate | 7 | 88.2 | 84.9 | 82.2 | 81.2 | 81.6 |
| F12 | Sodium Phosphate | 7.5 | 88.4 | 84.7 | 81.7 | 80.2 | 79.6 |
| F13 | None - Control 1 (Glycine) | 5.2 | 90.6 | 88.2 | 87.7 | 87 | 83.4 |
| F14 | None - Control 2 (Glycine) | 5.2 | 90.5 | 88.2 | 87.7 | 87 | 83.2 |

TABLE 17

Impact of Temperature Stress over Time on dimer stability with 5% Sorbitol

| | | | T=0 | T = 12 weeks | | | |
|---|---|---|---|---|---|---|---|
| Form. No. | Buffer | pH | | -70C | 5C | 25C | 40C |
| | | | Dimer (%) | Dimer (%) | Dimer (%) | Dimer (%) | Dimer (%) |
| F2 | Sodium Acetate | 4.7 | 8 | 8.9 | 9.4 | 8.9 | 7.6 |
| F4 | Sodium Acetate | 5 | 9.4 | 10.7 | 11.6 | 11.6 | 10 |
| F6 | Histidine | 6 | 11 | 13.4 | 15.1 | 15.6 | 13.1 |
| F8 | Sodium Phosphate | 6 | 10.2 | 12.6 | 13.9 | 14.3 | 12.2 |
| F10 | Sodium Phosphate | 7 | 11.2 | 14.1 | 16.3 | 17.1 | 14.6 |
| F12 | Sodium Phosphate | 7.5 | 10.9 | 14.3 | 16.7 | 17.8 | 15.1 |
| F13 | None - Control 1 (Glycine) | 5.2 | 8.9 | 11.1 | 11.5 | 12.1 | 12.1 |
| F14 | None - Control 2 (Glycine) | 5.2 | 9 | 11.1 | 11.5 | 12.1 | 12.1 |

### Example 9: Stability of N-Glycan and % Sialylation and Disialylation in Sorbitol Formulation of hsIgG with Various Buffers.

Sample formulations post filtration were transferred to glass vials, and stored at stress temperatures (-70° C., 5° C., 25° C., or 40° C.) over twelve weeks. Following storage at stress temperature, formulations were tested for purity by HILIC-HPLC. At time zero, all formulations displayed comparable A2F peak percentages (between 68.4% to 68.8%). The results are shown in Tables 18-21.

Storage at -70° C. and 5° C. resulted in comparable A2F peak percentages (70% to 70.9% for the former, and 69.9% to 70.4% for the latter), total sialylation (99.5% to 99.7%) monosialylation percentage (6.8% to 8.1%), and disialylated percentage (91.5% to 92.9%).

At 25° C., F2 exhibited decreases in A2F; however, it was not as significant as its glycine counterpart formulation. All other formulations at pH 5.0 and above displayed slight increases in peak A2F.

At 40° C., F2 did not exhibited significant changes in A2F, percent total sialylation, percent monosialylation, and percent disialylation peak as seen to its glycine counterpart at a similar pH. F4, sodium acetate formulation, was comparable to the controls with very minimal decrease in A2F peaks and percent total sialylation; and increases in percent monosialylation, and comparable decreases in percent disialylation.

Formulations at higher pH showed the least amount of change in sialylation, while formulations at the lowest pH show the most change.

TABLE 18

A2F Glycan Stability over Temperature Stresses

| | | | T = 0 | T = 12 weeks | | | |
|---|---|---|---|---|---|---|---|
| Form. No. | Buffer | pH | | -70C | 5C | 25C | 40C |
| | | | A2F | A2F | A2F | A2F | A2F |
| F2 | Sodium Acetate | 4.7 | 68.6 | 70.3 | 69.9 | 68.2 | 55.7 |
| F4 | Sodium Acetate | 5 | 68.7 | 70.3 | 70 | 69.5 | 62.6 |
| F6 | Histidine | 6 | 68.4 | 70.5 | 70.1 | 69.9 | 68 |
| F8 | Sodium Phosphate | 6 | 68.5 | 70.3 | 70.2 | 69.7 | 66.5 |
| F10 | Sodium Phosphate | 7 | 68.6 | 70.4 | 69.9 | 69.8 | 69.3 |
| F12 | Sodium Phosphate | 7.5 | 68.6 | 70.9 | 70.2 | 70.1 | 69.8 |
| F13 | None - Control 1 (Glycine) | 5.2 | 68.6 | 70.4 | 70.4 | 69.3 | 62.7 |
| F14 | None - Control 2 (Glycine) | 5.2 | 68.8 | 70 | 70.1 | 69.2 | 62.7 |

TABLE 19

Total Percent Sialylation Stability over Temperature Stresses

| | | | T = 0 | T = 12 weeks | | | |
|---|---|---|---|---|---|---|---|
| | | | | -70C | 5C | 25C | 40C |
| Form. No. | Buffer | pH | Total Sialylation (% Area) | Total Sialylation (% Area) | Total Sialylation (% Area) | Total Sialylation (% Area) | Total Sialylation (% Area) |
| F2 | Sodium Acetate | 4.7 | 99.5 | 99.6 | 99.6 | 99.5 | 98.5 |
| F4 | Sodium Acetate | 5 | 99.5 | 99.6 | 99.6 | 99.6 | 99.3 |
| F6 | Histidine | 6 | 99.5 | 99.6 | 99.6 | 99.6 | 99.6 |
| F8 | Sodium Phosphate | 6 | 99.5 | 99.6 | 99.6 | 99.6 | 99.5 |
| F10 | Sodium Phosphate | 7 | 99.5 | 99.6 | 99.6 | 99.6 | 99.6 |
| F12 | Sodium Phosphate | 7.5 | 99.5 | 99.7 | 99.6 | 99.6 | 99.6 |
| F13 | None -Control 1 (Glycine) | 5.2 | 99.5 | 99.6 | 99.6 | 99.6 | 99.2 |
| F14 | None -Control 2 (Glycine) | 5.2 | 99.5 | 99.6 | 99.6 | 99.5 | 99.2 |

TABLE 20

Monosialylation Stability over Temperature Stresses

| | | | T = 0 | T = 12 weeks | | | |
|---|---|---|---|---|---|---|---|
| | | | | -70C | 5C | 25C | 40C |
| Form. No. | Buffer | pH | Mono-sialylated (% Area) | Mono-sialylated (% Area) | Mono-sialylated (% Area) | Mono-sialylated (% Area) | Mono-sialylated (% Area) |
| F2 | Sodium Acetate | 4.7 | 8 | 7.5 | 7.7 | 9.7 | 26 |
| F4 | Sodium Acetate | 5 | 7.9 | 7.4 | 7.6 | 8 | 16.8 |
| F6 | Histidine | 6 | 8 | 7.1 | 7.5 | 7.8 | 9.8 |
| F8 | Sodium Phosphate | 6 | 8 | 7.3 | 7.3 | 7.7 | 12.1 |
| F10 | Sodium Phosphate | 7 | 8.1 | 7.4 | 7.8 | 7.7 | 7.9 |
| F12 | Sodium Phosphate | 7.5 | 8 | 6.8 | 7.3 | 7.2 | 7.6 |
| F13 | None -Control 1 (Glycine) | 5.2 | 8 | 7.3 | 7 | 8.4 | 16.7 |
| F14 | None -Control 2 (Glycine) | 5.2 | 7.9 | 7.3 | 7.5 | 8.3 | 16.9 |

TABLE 21

Disialylation Stability over Temperature Stresses

| | | | T = 0 | T = 12 weeks | | | |
|---|---|---|---|---|---|---|---|
| | | | | -70C | 5C | 25C | 40C |
| Form. No. | Buffer | pH | Di-sialylated (% Area) | Di-sialylated (% Area) | Di-sialylated (% Area) | Di-sialylated (% Area) | Di-sialylated (% Area) |
| F2 | Sodium Acetate | 4.7 | 91.7 | 92.1 | 91.9 | 89.8 | 72.5 |
| F4 | Sodium Acetate | 5 | 91.5 | 92.2 | 91.9 | 91.5 | 82.5 |
| F6 | Histidine | 6 | 91.6 | 92.5 | 92.1 | 91.8 | 89.8 |
| F8 | Sodium Phosphate | 6 | 91.6 | 92.3 | 92.2 | 91.9 | 87.4 |
| F10 | Sodium Phosphate | 7 | 91.6 | 92.2 | 91.8 | 91.9 | 91.7 |
| F12 | Sodium Phosphate | 7.5 | 91.6 | 92.9 | 92.2 | 92.4 | 92 |
| F13 | None - Control 1 (Glycine) | 5.2 | 91.5 | 92.3 | 92.5 | 91.1 | 82.5 |
| F14 | None - Control 2 (Glycine) | 5.2 | 91.5 | 92.2 | 92.1 | 91.2 | 82.3 |

### Example 10: Charge Variants Within Stabilized Sorbitol Formulation of hsIgG with Various Buffers at Various Stress Temperatures

Sample formulations post filtration were transferred to glass vials and stored at stress temperatures (-70° C., 5° C., 25° C., or 40° C.) over twelve weeks. Following storage at stress temperature, formulations were tested for changes in charge-variants and isoelectric peaks by imaged capillary isoelectric focusing (icIEF) to understand formulation stability at different stress temperatures.

At time zero, all formulations exhibited similar acid, neutral and basic combined peak percentages, as well as a main peak isoelectric point near 7.4. All main peak isoelectric points remained around 7.4 regardless of temperature stress. The results are shown in Tables 22-25.

Formulations exhibited negligible changes in isoform percentages with no significant trends observed at -70° C. and 5° C.

At 25° C., F2 and F4 formulations showed comparable acidic, neutral and basic peak percentages to the control formulations, with negligible changes from time zero (<1%). At 25° C., all formulations (except F14) displayed slight decreases in basic peak percentages, with corresponding increases across neutral and acidic percentages. The decrease is basic peak percentages were lower than their glycine counterparts.

At 40° C., formulations F2 and F4 showed the least amount of change in peak percentages and were comparable to the control formulations. All formulations displayed decreases in basic peak percentage. All sorbitol formulations exhibit an increase in acidic peak percentages and neutral peak percentages, unlike their glycine counterparts. Formulations at higher pH show the most significant changes compared to time zero, and sorbitol formulations show less changes than their glycine counterparts.

TABLE 22

Acidic Charge Variant changes over Temperature Stresses

| | | T = 0 | T = 12 weeks | | | |
|---|---|---|---|---|---|---|
| | | | -70C | 5C | 25C | 40C |
| Form. No. | Buffer | Acidic (%) | Acidic (%) | Acidic (%) | Acidic (%) | Acidic (%) |
| F2 | Sodium Acetate | 23.58 | 23.63 | 23.71 | 24.24 | 23.78 |
| F4 | Sodium Acetate | 24.39 | 24.37 | 24.19 | 24.77 | 25.05 |
| F6 | Histidine | 24.02 | 24.3 | 24.24 | 24.36 | 26.73 |
| F8 | Sodium Phosphate | 24.08 | 23.6 | 24.64 | 24.54 | 26.24 |
| F10 | Sodium Phosphate | 24.69 | 23.47 | 24.29 | 24.86 | 28.86 |
| F12 | Sodium Phosphate | 24.17 | 25.28 | 24.42 | 25.49 | 32.78 |
| F13 | None - Control 1 | 23.5 | 24.36 | 23.64 | 24.34 | 25.51 |
| F14 | None - Control 2 | 23.74 | 23.56 | 23.51 | 23.78 | 25.61 |

TABLE 23

Neutral Charge Variant changes over Temperature Stresses

| | | T = 0 | T = 12 weeks | | | |
|---|---|---|---|---|---|---|
| | | | -70C | 5C | 25C | 40C |
| Form. No. | Buffer | Neutral (%) | Neutral (%) | Neutral (%) | Neutral (%) | Neutral (%) |
| F2 | Sodium Acetate | 32.46 | 32.77 | 32.3 | 32.53 | 33.75 |
| F4 | Sodium Acetate | 32.44 | 33.3 | 32.57 | 32.91 | 33.93 |
| F6 | Histidine | 33.49 | 34.09 | 34.35 | 33.83 | 34.43 |
| F8 | Sodium Phosphate | 32.04 | 33.1 | 33.91 | 32.92 | 33.85 |
| F10 | Sodium Phosphate | 33.08 | 34.04 | 33.86 | 33.74 | 33.94 |
| F12 | Sodium Phosphate | 32.78 | 34.59 | 34.06 | 33.35 | 33.46 |
| F13 | None - Control 1 | 32.42 | 32.8 | 32.5 | 32.01 | 33.7 |
| F14 | None - Control 2 | 32.64 | 32.19 | 32.48 | 32.15 | 33.98 |

TABLE 24

Basic Charge Variant changes over Temperature Stresses

| | | T = 0 | T = 12 weeks | | | |
|---|---|---|---|---|---|---|
| | | | -70C | 5C | 25C | 40C |
| Form. No. | Buffer | Basic (%) | Basic (%) | Basic (%) | Basic (%) | Basic (%) |
| F2 | Sodium Acetate | 43.96 | 43.6 | 44 | 43.23 | 42.47 |
| F4 | Sodium Acetate | 43.17 | 42.32 | 43.24 | 42.31 | 41.02 |
| F6 | Histidine | 42.49 | 41.62 | 41.41 | 41.81 | 38.83 |
| F8 | Sodium Phosphate | 43.88 | 43.3 | 41.46 | 42.54 | 39.91 |
| F10 | Sodium Phosphate | 42.23 | 42.49 | 41.85 | 41.4 | 37.2 |
| F12 | Sodium Phosphate | 43.05 | 40.13 | 41.52 | 41.16 | 33.76 |
| F13 | None - Control 1 | 44.07 | 42.84 | 43.86 | 43.66 | 40.78 |
| F14 | None - Control 2 | 43.62 | 44.25 | 44.01 | 44.07 | 40.4 |

TABLE 25

Main peak isoelectric changes over temperature stresses

| | | T = 0 | T = 12 weeks | | | |
|---|---|---|---|---|---|---|
| | | | -70C | 5C | 25C | 40C |
| Form. No. | Buffer | Main Peak (pI) | Main Peak (pI) | Main Peak (pI) | Main Peak (pI) | Main Peak (pI) |
| F2 | Sodium Acetate | 7.4 | 7.42 | 7.41 | 7.4 | 7.41 |
| F4 | Sodium Acetate | 7.4 | 7.41 | 7.41 | 7.4 | 7.4 |
| F6 | Histidine | 7.39 | 7.41 | 7.41 | 7.4 | 7.41 |
| F8 | Sodium Phosphate | 7.39 | 7.41 | 7.41 | 7.4 | 7.4 |
| F10 | Sodium Phosphate | 7.39 | 7.42 | 7.41 | 7.4 | 7.4 |
| F12 | Sodium Phosphate | 7.4 | 7.4 | 7.4 | 7.4 | 7.4 |
| F13 | None - Control 1 | 7.39 | 7.42 | 7.42 | 7.41 | 7.41 |
| F14 | None - Control 2 | 7.4 | 7.42 | 7.42 | 7.42 | 7.41 |

### Example 11: High Concentration Formulation

Six formulations of M254 hsIgG containing 10 mM sodium acetate, 5% (w/v) sorbitol, and 0.02% (w/v) polysorbate 20 at pH 5.3 were prepared at various concentrations of IgG (100 mg/mL, 125 mg/mL, 175 mg/mL, 200 mg/mL, 250 mg/mL, and 275 mg/mL). A control was also prepared with 100 mg/mL IgG, 250 mM glycine, 0.02% (w/v) polysorbate 20 at pH 5.2.

The formulations were stored at 5° C., 25° C., and 40° C. Visual appearance, pH, concentration (A₂₈₀), turbidity (A₆₅₀), size exclusion chromatography (SEC), and UNCLE (Unchained Labs) were characterized pre-fill and at one week. UNCLE was used to evaluate aggregation curves and determine temperature of aggregation using scattering light intensity at 473 nm over a thermal ramp from 20° C. to 90° C. Osmolality was tested on pre-fill samples. Viscosity was tested at time zero (after filtration) and at one week. There were no changes in visual appearance (all formulations at different storage conditions remained clear, colorless and free of visible particles), pH (Table 26), or turbidity (Table 27) after storage at different temperatures for one week. Concentration decreased at the one week timepoint for all formulations (Table 28). Osmolality increased with increasing hsIgG concentration (Table 29), and viscosity increased with increasing hsIgG concentration (Table 29; Table 30). No significant soluble aggregate increases were observed with increased concentration. Across all storage temperatures, 100 mg/mL and 125 mg/mL hsIgG samples maintained slightly higher monomer percentages compared to samples at 175 mg/mL and above (Table 31). UNCLE data showed concentration increase only slightly reduces stability of the product, with lower Tagg (FIG. 3).

TABLE 26

Results-pH

| Sample Name* | pH | | | |
|---|---|---|---|---|
| | Pre-Fill | T=1 week (5° C.) | T=1 week (25° C.) | T=1 week (40° C.) |
| 100 mg/mL | 5.30 | 5.31 | 5.26 | 5.23 |
| 125 mg/mL | 5.34 | 5.31 | 5.29 | 5.28 |
| 175 mg/mL | 5.40 | 5.39 | 5.36 | 5.32 |
| 200 mg/mL | 5.40 | 5.40 | 5.37 | 5.37 |
| 250 mg/mL | 5.41 | 5.41 | 5.39 | 5.40 |
| 275 mg/mL | 5.37 | 5.44 | 5.43 | 5.42 |

*Sample names are based on pre-fill concentrations

TABLE 27

Results-Turbidity

| Sample Name* | Turbidity (A₆₅₀) | | | |
|---|---|---|---|---|
| | Pre-Fill | T=1 week (5° C.) | T=1 week (25° C.) | T=1 week (40° C.) |
| 100 mg/mL | 0.003 | 0.002 | 0.001 | 0.002 |
| 125 mg/mL | 0.001 | 0.001 | 0.004 | 0.002 |
| 175 mg/mL | 0.004 | 0.001 | 0.002 | 0.002 |
| 200 mg/mL | 0.002 | 0.001 | 0.002 | 0.001 |
| 250 mg/mL | 0.003 | 0.001 | 0.001 | 0.001 |
| 275 mg/mL | 0.001 | 0.002 | 0.002 | 0.002 |

*Sample names are based on pre-fill concentrations

TABLE 28

Results-Concentration

| Sample Name* | Concentration (mg/mL) by A₂₈₀ | | | |
|---|---|---|---|---|
| | Pre-Fill | T=1 week (5° C.) | T=1 week (25° C.) | T=1 week (40° C.) |
| 100 mg/mL | 100.4 | 89.5 | 90.6 | 91.0 |
| 125 mg/mL | 128.6 | 86.5 | 92.9 | 101.9 |
| 175 mg/mL | 176.9 | 157.1 | 170.6 | 160.8 |
| 200 mg/mL | 204.3 | 156.3 | 167.9 | 160.8 |
| 250 mg/mL | 246.1 | 183.5 | 182.9 | 206.8 |
| 275 mg/mL | 276.5 | 213.4 | 544.5 | 252.9 |

*Sample names are based on pre-fill concentrations

TABLE 29

Results-Osmolality and Viscosity

| IgG Concentration (at pre-fill) | Osmolality (mOsm/kg) | Viscosity (cP) |
|---|---|---|
| 100 mg/mL | 347 | 2.62 |
| 125 mg/mL | 374 | 2.55 |
| 175 mg/mL | 413 | 11.34 |
| 200 mg/mL | 470 | 7.54 |
| 250 mg/mL | 508 | 30.43 |
| 275 mg/mL | Sample failed to freeze | 57.86 |

*Sample names are based on pre-fill concentrations

TABLE 30

Results-Time Zero Viscosity v. Average Concentration at 1 week

| Sample Name* | Average Concentration @ T=1 week (mg/mL) | Average Viscosity @ T=0 (cP) |
|---|---|---|
| 100 mg/mL | 90.4 | 2.62 |
| 125 mg/mL | 93.8 | 2.55 |
| 175 mg/mL | 162.8 | 11.34 |
| 200 mg/mL | 161.7 | 7.54 |
| 250 mg/mL | 191.1 | 30.43 |
| 275 mg/mL | 236.9 | 57.86 |

*Sample names are based on pre-fill concentrations

TABLE 31

Results-SEC

| Sample Name* | T=0 % Dimer | T=0 % Monomer | T=1w; 5° C. % Dimer | T=1w; 5° C. % Monomer | T=1w; 55° C. % Dimer | T=1w; 25° C. % Monomer | T=1w; 40° C. % Dimer | T=1w; 40° C. % Monomer |
|---|---|---|---|---|---|---|---|---|
| DS (5° C. Avg) | 9.5 | 89.3 | 13.2 | 85.5 | 13.2 | 85.5 | 13.2 | 85.5 |
| 100 mg/mL | 10.1 | 88.7 | 8.0 | 91.0 | 7.4 | 91.9 | 6.2 | 93.0 |
| 125 mg/mL | 9.9 | 88.9 | 7.8 | 91.2 | 7.1 | 92.1 | 6.4 | 92.9 |
| 175 mg/mL | 10.8 | 88.0 | 8.5 | 90.6 | 8.0 | 91.0 | 7.2 | 91.9 |
| 200 mg/mL | 10.4 | 88.1 | 8.5 | 9037 | 7.9 | 91.0 | 7.2 | 92.1 |
| 250 mg/mL | 10.6 | 88.1 | 8.3 | 90.6 | 8.0 | 91.0 | 7.7 | 91.6 |
| 275 mg/mL | 10.6 | 88.1 | 8.4 | 90.7 | 8.4 | 90.6 | 7.9 | 91.2 |

*Sample names are based on pre-fill concentrations

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A liquid pharmaceutical composition comprising immunoglobulins in about 10 mM sodium acetate, about 0.02% (w/v) polysorbate 20, and at least one of about 250 mM glycine or about 5% (w/v) sorbitol, wherein at least 50% of branched glycans on the Fc region of the immunoglobulins are disialylated by way of NeuAc-α 2,6-Gal terminal linkages, wherein the pH of the composition is 4 - 7.

2. The liquid pharmaceutical composition of claim 1 comprising 250 mM glycine.

3. The liquid pharmaceutical composition of claim 1 comprising 5% (w/v) sorbitol.

4. The liquid pharmaceutical composition of claim 1, wherein the concentration of immunoglobulins is 50-275 mg/mL.

5-7. (canceled)

8. The liquid pharmaceutical composition of claim 1, wherein at least 60%, 70%, 80%, 90% or 95% of branched glycans on the Fc domain of the immunoglobulins are disialylated by way of NeuAc-α 2,6-Gal terminal linkages.

9-12. (canceled)

13. The liquid pharmaceutical composition of claim 1, wherein 5-20% of the immunoglobulins are dimers.

14. The liquid pharmaceutical composition of claim 13, wherein 5-10% of the immunoglobulins are dimers.

15. The liquid pharmaceutical composition of claim 1, wherein at least 80% of the immunoglobulins are monomers or dimers.

16. The liquid pharmaceutical composition of claim 15, wherein at least 85% of the immunoglobulins are monomers or dimers.

17. The liquid pharmaceutical composition of claim 16, wherein at least 90% of the immunoglobulins are monomers or dimers.

18-30. (canceled)

30. The liquid pharmaceutical composition of claim 1, wherein the composition has fewer than 1000 particles with a diameter between 10 and 100 micrometers after agitation at 1000 RPM for 8 hours at 2-8° C.

31-38. (canceled)

39. The liquid pharmaceutical composition of claim 1, wherein the formulation is stable at 5° C. for at least 7 months, at 25° C. for at least one month, 2-8° C. for two years, and/or two weeks at 15-30° C.

40-43. (canceled)

44. A liquid pharmaceutical composition comprising immunoglobulins in about 10 mM sodium acetate, about 0.02% (w/v) polysorbate 20, and at least one of about 250 mM glycine or about 5% (w/v) sorbitol, wherein at least 50% of branched glycans on the immunoglobulins are disialylated by way of NeuAc- α 2,6-Gal terminal linkages, wherein the pH of the composition is 4 - 7.

45. The liquid pharmaceutical composition of claim 44 comprising 250 mM glycine.

46. The liquid pharmaceutical composition of claim 44 comprising 5% (w/v) sorbitol.

47. The liquid pharmaceutical composition of claim 44, wherein the concentration of immunoglobulins is 50-275 mg/mL.

48-86. (canceled)

87. A method for treating a disorder, the method comprising administering the liquid pharmaceutical composition of any of the forgoing claims at a dose that is 1%-10% of the effective dose of IVIG for treating the disorder.

88. The method of claim 87, wherein the hsIgG preparation is administered at a dose of 5 mg/kg to 100 mg/kg.

89. The method of claim 87, wherein the disorder is an inflammatory disorder.

90. The method of claim 87, wherein the subject is suffering from antibody deficiency.

91-136. (canceled)
